(19) United States
(12) Patent Application Publication
(10) Pub. No.: US 2024/0277252 A1
(43) Pub. Date: Aug. 22, 2024

(54) MULTIPLEXED ANTIGEN-BASED DETECTION OF SARS-COV-2 AND OTHER DISEASES USING NANOMECHANICAL SENSORS

(71) Applicant: Northwestern University, Evanston, IL (US)

(72) Inventors: Vinayak P. Dravid, Evanston, IL (US); Gajendra S. Shekhawat, Evanston, IL (US)

(21) Appl. No.: 18/572,345

(22) PCT Filed: Jun. 22, 2022
(86) PCT No.: PCT/US22/34607

§ 371 Date: Dec. 20, 2023

Related U.S. Application Data
(60) Provisional application No. 63/202,713, filed on Jun. 22, 2021.

(51) Int. Cl. A61B 5/08; A61B 5/097; A61B 10/00; B81B 7/04; G01N 33/543; G01N 33/569

(52) U.S. Cl. CPC A61B 5/082; A61B 5/097; B81B 7/04; G01N 33/54373; G01N 33/56983; A61B 2010/0087; B81B 2201/0214; B81B 2203/0118; G01N 2333/165; G01N 2469/10; G01N 2469/20

(57) ABSTRACT

Nanomechanical sensors comprising an antibody-functionalized microcantilever and methods of using the same are described herein.

### CROSS-REFERENCE TO RELATED APPLICATIONS

The present application claims priority to U.S. Provisional Patent Application No. 63/202,713, filed Jun. 22, 2021, the entire contents of which are hereby incorporated by reference.

### STATEMENT REGARDING FEDERALLY SPONSORED RESEARCH

This invention was made with government support under HL119810 awarded by National Institutes of Health. The government has certain rights in the invention.

### BACKGROUND OF THE INVENTION

The outbreak of Severe Acute Respiratory Syndrome Coronavirus-2 (SARS-CoV-2) has resulted in, as of Apr. 7, 2021, over 132 million cases worldwide including 1.96 million deaths. Given the long incubation period of COVID-19 (2-14 days) and its highly contagious nature, the early and large-scale screening of COVID-19 is of paramount importance to reduce viral transmission. Since the FDA EUA approval of real-time RT-PCR for molecular diagnosis of COVID-19, it has been utilized as the ‘gold standard’ for SARS-CoV-2 detection. However, RT-PCR suffers from long turnaround time due to complicated sample preparation and laboratory analysis which affects the diagnostic accuracy. In addition, the need for specialized instruments to perform the test and specialists to operate the equipment makes it un-reachable in resource-poor settings. Immunoassays to detect SARS-CoV-2 antigens are an alternative to PCR-based testing approaches, and they offer significant potential benefits. There are antigen tests available, but they often suffer from higher false positives and false negatives rates. Thus, the inaccurate tests are in fact detriment to disease spread and its management. Hence, there continues to be an urgent need for a rapid, cost-effective, point-of-care (POC) antigen detection tests that can directly detect SARS-CoV-2 immunogenic proteins with high sensitivity and specificity even before the onset of symptoms.

### BRIEF SUMMARY OF THE INVENTION

Nanomechanical sensors comprising an antibody-functionalized microcantilever and methods of using the same are described. The antibody-functionalized microcantilever may comprise a microcantilever, an antibody configured to bind a target antigen or an antigen configured to bind a target antibody, and a tether covalently tethering the antibody or the antigen to the microcantilever. The microcantilever has an effective surface density of tethered antibodies or antigens thereon, which allows for the detections of antigens or antibodies present in a sample. In some embodiments, the antibody-functionalized microcantilever comprises an antibody configured to bind a pathogenic antigen or an antibody indicative of an infection. The sensors described herein may be functionalized to detect a pulmonary or respiratory disease, such as SARS-CoV-2 infection. In some embodiments, the sensors comprise a field-effect transistor, such as a MOSFET, which allows for electrical detection of antibody-antigen binding.

Systems comprising the nanomechanical sensors are also described. The systems comprise a nanomechanical sensor and a detector for detecting a response of the nanomechanical sensor to the sample and generating a signal indicative of the response of the nanomechanical sensor to the sample. Suitably, the detector is configured to detect a response such as a bending moment. The systems described herein may comprise two or more different nanomechanical sensors, thereby allowing for multiplexed detection. The different sensors may be functionalized with different antibodies, different antigens, or both an antibody and antigen. In some embodiments, the system comprises a breath collector, thereby allowing for noninvasive sample collection.

Methods of detecting a target antigen or antibody in a sample are also provided. The method may comprise contacting a nanomechanical sensor with the sample and detecting for a response of the antibody-functionalized microcantilever to the sample. The methods described herein allow for rapid detection of the target. In some embodiments, the response to the target antigen or the target antibody is detected within 5 minutes of contacting the nanomechanical sensor with the sample. The methods also allow for sensitive detection. In some embodiments, the sample comprises less than 10 ng/ml of the target antigen or the target antibody.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1: Optical detection scheme of SARS-CoV-2 S1 (RBD-Receptor Binding Domain) protein on a microcantilever surface

FIG. 2: Schematic of cantilever biofunctionalization using EDC-NHS chemistry. Monoclonal antibodies for spike and nucleocapsid proteins were immobilized on functionalized microcantilever surface.

FIG. 3: Real-time detection for different concentrations of SARS-CoV-2 S1 antigen protein; microcantilever deflection for different concentrations of S1 protein was measured onto anti-S1 antibody coated microcantilevers (a) Deflection plot for 1 μg/ml of antigen concentration, (b) Deflection plot for 100 ng/ml of concentration, (c) Deflection plot for 10 ng/ml of concentration, (d) Deflection plot for 1 ng/ml of concentration.

FIG. 4: Real-time response for different concentrations of SARS-CoV-2 Nucleocapsid (N) protein; microcantilever deflection for different concentrations of N protein was measured onto anti-N antibody coated microcantilevers, (a) Deflection plot for 1 μg/ml of antigen concentration, (b) Deflection plot for 100 ng/ml of concentration, (c) Deflection plot for 10 ng/ml of concentration, (d) Deflection plot for 1 ng/ml of concentration.

FIG. 5: Concentration dependent response curves for SARS-CoV-2 S1 and N proteins; cross-validation and control experiments were also performed by interacting both S1 and N proteins in different combinations on the cantilever surface to ascertain the specificity in the detection, (a) Quantification plots for S1 protein detection, (b) Quantification plots for N protein detection. Anti-S/N protein: detection of N protein onto an anti-S1 antibody conjugated cantilever (cross-validation); Anti-S/N-S mix: interaction of N and S1 protein mixture solution onto an anti-S1 antibody coated cantilever surface (cross-validation); anti-N/S protein: detection of S1 protein onto an anti-N antibody conjugated cantilever (cross-validation); anti-N/S-N mix: interaction of S1 and N protein mixture solution onto an anti-N antibody conjugated cantilever surface.

FIG. 6: Optical deflection measurement on patient samples with different Ct values. The positive sample with the highest Ct value (39.7) yielded the lowest deflection (8.46+/−3-4 nm) whereas maximum deflection (48.91+/−3-4 nm) was exhibited for the patient sample having the lowest Ct value (13.18).

FIG. 7: Binding study of mutant UK variant spike protein on the cantilever surface. Prior to this, all cantilevers were biofunctionalized with anti-spike (RBD) antibody.

FIG. 8: Optimization of antibody coverage on cantilever surface for deflection measurements. Prior to antibody attachment, all cantilevers were first functionalized with EDC-NHS chemistry, (a) Surface coverage plot for different concentrations of SARS-CoV-2 anti-S1 (RBD) antibody, (b) Cantilever deflection Vs antibody concentration plot.

FIG. 9: SEM micrograph of a microcantilever device

FIG. 10: Complete biofunctionalization process of the microcantilever sensors towards SARS-CoV-2 detection

FIG. 11: Transduction mechanism (right) and 2D micro fabricated cantilever arrays (left) with on-chip MOSFET detection. Panel (a) shows a SEM micrograph of integrated MOSFET cantilever arrays and Panel (b) shows schematic depicting the active and reference cantilever for differential readout.

FIG. 12: Schematic of breath collection set-up with integrated detection chamber.

FIG. 13: SARS-CoV-2 detection through drain-current experiments from patients' nasopharyngeal swab samples, Panel (a) shows current modulation experiments on cantilevers coated with SARS-CoV-2 nucleocapsid (N) antibody, Panel (b) shows measurements on cantilevers coated with spike (S1) antibody, Panel (c) shows another set of data for COVID sensing using 20 patients' samples on anti-N coated cantilevers. Here data collected from gold coated cantilevers (without any target interaction) considered as a control sample. The level of infectivity of these patients were pre-determined with respect to the Ct values using qRT-PCR method. For N-antibody coated cantilevers, the shift in the drain current was large in magnitude in comparison with anti-S coated cantilevers. For positive patients' samples, a clear shift in the drain current (spacing between the curves) can be observed whereas all the negative samples are clumped together indicating no significant change in the current.

FIG. 14: Breath based detection of COVID through current modulation (Id-Vd) measurement of the MOSFET device, Panel (a) shows Id-Vd measurement data from 24 positive patients, panel (b) shows Id-Vd measurement data from COVID positive and negative patients, panel (c) shows dynamic representation of the Id-Vd measurements (sensing with respect to the time), panel (d) shows schematic showing the developed COVID test by stratification into green zone and red zone. In the first set of data, breath samples were collected from 24 COVID positive patients along with a control (measurement from reference cantilever). In another study, twenty COVID patients volunteered for the breath samples out of which ten were positive and ten were negative. The level of infectivity of these patients were pre-determined with respect to the Ct values using qRT-PCR method. For positive patients, a clear shift in the drain current (spacing between the curves) can be observed whereas all the negative samples are clumped together indicating no significant change in the drain current.

FIG. 15: Multiplexed detection of different breath exhaled viral antigens through I_{d}-V_{d} measurements. Panel (a) shows breath detection of SARS-CoV-2 Delta variant, Panel (b) shows detection of Influenza A virus, Panel (c) shows detection of Enterovirus in patients' breath, panel (d) shows HIN1 (Swine flu) detection, and panel (e) shows detection of Pneumonia parasite. Individual cantilevers were inserted inside the capillary channel filled with the corresponding antibody facilitating receptor coatings on all the cantilevers and those bio-functionalized chips were then attached to the breath detection set-up. The control represents here the cantilever device which was not coated with any antibody. The SARS-CoV-2 Delta variant exhibited maximum change in the drain current (I_{d}=70-78 μA) among all the patients volunteered indicating higher viral load in the upper respiratory track. Inset pictures shows the zoomed version of the I_{d}-V_{d} plots just to demonstrate the better separation in the curves.

FIG. 16: Serological testing data (I-V) from the patients' samples collected after three weeks of recovery from COVID-19. A total of twenty persons (n=20) volunteered for this study out of which ten were SARS-CoV-2 positive and ten were negative. The patients were screened for antibodies against the nucleocapsid protein and corresponding antigens were coated on the devices using SATA chemistry. Samples collected from the patients with higher viral load (higher antibody titer) demonstrated a large change in the current in comparison with the patients with lower viral load exhibiting low level of antibody concentration. The data from the negative samples can be seen clumped together in a single curve depicting no change in the current.

FIG. 17: MOSFET based detection of SARS-CoV-2 S1 protein, Panels (a-c) show different set of experiments using four different concentrations of S1 protein, panel (d) shows S1 protein detection with a reference cantilever. MOSFET devices were first biofunctionalized by EDC-NHS chemistry to anchor anti-S1 antibody on the surface. Decrease in channel conductance (decreased current) with the increasing S1 concentrations abbreviated to compressive surface stress (repulsive forces). Panels (a-c) show the control sample denotes to data from S1 coated cantilever where antibody conjugation was not carried out. Reference cantilever denotes to SiN (Silicon nitride with no gold layer) cantilever without any antibody immobilization step. Panel (d) exhibits no significant change in the drain current for reference cantilever (no separation between the curves for different N concentrations).

FIG. 18: MOSFET based detection of SARS-CoV-2 nucleocapsid (N) protein. MOSFET devices were first biofunctionalized by EDC-NHS chemistry to anchor anti-N-antibody on the surface. The increased concentrations of N protein resulted in a decreased current. The gold sample curve denotes to antibody conjugated gold coated cantilever (but no antibody-antigen interaction) and SiN denotes to silicon nitride cantilever with no gold coating (reference). The cantilevers with gold and diluted PBS buffer did not show any significant change in the current. The reference cantilever (SiN) did not yield any current change for all the concentrations of N proteins.

FIG. 19: MOSFET sensor array as a switch for SARS-CoV-2 detection (schematic). The figure represents the change in the drain current as a switch (threshold window) where green dots show no change in the drain current for negative samples and red color demonstrates the shift in the drain current with respect to different Ct values (current inversely proportional to the Ct values)

FIG. 20: Chip design for fabricated MOSFET device used for breath and swab-based detection of SARS-CoV-2

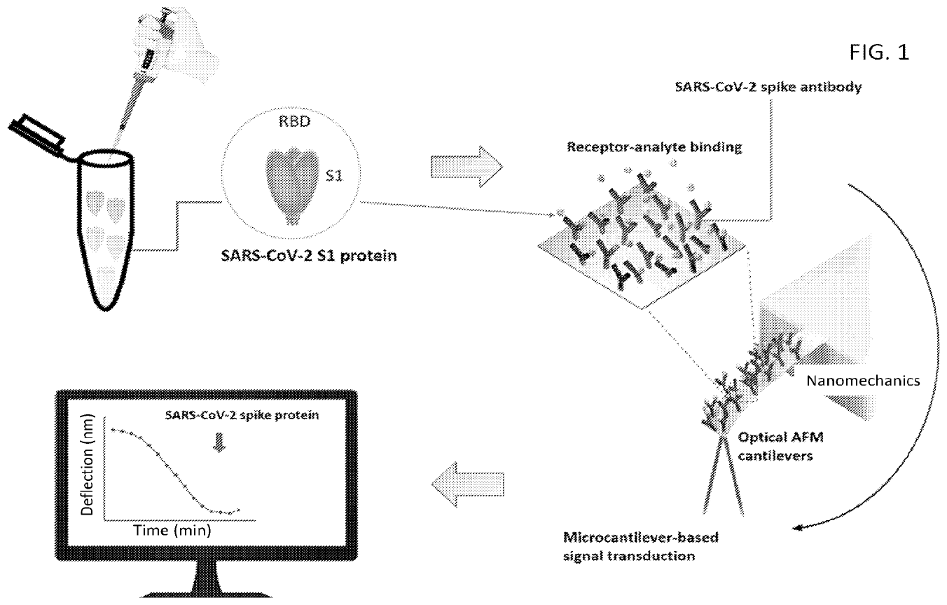

### DESCRIPTION OF THE INVENTION

Herein we report nanomechanical sensors, systems, and methods based on nanomechanical signal transduction of specific binding on top of microcantilevers that can readily and specifically detect targets in minutes. The Examples demonstrate rapid detection of SARS-CoV-2 nucleocapsid (N) and spike (S1) receptor binding domain (RBD) proteins of the virus at clinically relevant concentration by real-time monitoring of nanomechanical signaling induced by antibody-antigen binding. The Examples also demonstrate that results are available in less than 10 minutes between subject sampling and testing. Cross-corroborated with qRT-PCR, the extent of viral load in patient samples shows remarkable consistency with nanomechanical signaling. This allows for detection of weaker binding and lowered response, which may be commensurate with reduced binding interactions. Indeed, the Examples demonstrate weaker binding affinity of U.K. mutant variant against antibodies derived from the wild type antigens. Thus, integration and automation of microcantilever-based real-time diagnostic assay promises rapid, accurate and cost-effective detection of antigens, potentially mutant variants, and other specific-binding related phenomena.

Microcantilever-based sensor platforms offer low cost, high sensitivity, rapid response, and label-free detection system. The microcantilever sensor platforms work as a transducer for the translation of a biomolecular recognition phenomenon on its surface into a response signal. These microsensors may be integrated in an array inside a chip which makes them amenable for parallel detection of several analytes at a time.

One aspect of the invention provides for a nanomechanical sensor comprising an antibody-functionalized microcantilever. As used herein, “antibody-functionalized” means a microcantilever comprising a tethered antibody or tethered antigen where binding of an antigen in a sample to the tethered antibody or an antibody in a sample to the tethered antigen results in a detectable response. The Examples demonstrate the use of an antibody-functionalized microcantilever where an antibody is tethered to the microcantilever and a response is generated by binding of antigen in a sample to the tethered antibody, but the antigen and antibody may also be used.

The response may be a bending moment. A bending moment, means a reaction induced in the microcantilever when an external force or moment is applied to the microcantilever, causing the microcantilever to bend. In some instances, the response may be referred to as a deflection of the microcantilever.

The antibody-functionalized microcantilevers may be used to detect a target antigen or target antibody by detecting a response of the antibody-functionalized microcantilevers to a sample. Assaying samples for use in clinical and non-clinical settings is described below. It can be generally useful to quantitatively test a sample, such as a biofluid obtained from a subject. In some embodiments, the biofluid can include, without limitation, mucus, saliva, sputum, blood, serum, urine, cerebrospinal fluid, interstitial fluid, and other fluid samples. In some embodiments, the sample is obtained by nasopharyngeal swab. In another embodiment, the sample is obtained by an exhalation. The systems described herein generally operate by detecting the presence or absence of a target antigen or antibody. This may be accomplished by binding the target antigen or the antibody to the antibody-functionalized microcantilever such that a response can be detected with a detector.

Viral and antigen loads in breath tend to be lower compared to saliva and/or nasopharyngeal swabs. The average viral load in the patients' exhaled breath is of the order of 10²-10⁴ viral particles compared to nasopharyngeal swab samples (˜10⁶-10⁸) from the same patients. Thus, for successful breath sampling, the detection device must exhibit not only ultra-high sensitivity but also specificity for the antigens across other analytes as well as selectivity amidst other proteins and markers. The nanomechanical sensors described herein fulfill these stringent requirements and the Examples demonstrate that the sensors have excellent sensitivity for breath sampling, compatibility with multiplexing and are amenable to system integration that can be cost effective and widely deployable.

As used herein, “subject” or “patient” refers to mammals and non-mammals. A “mammal” may be any member of the class Mammalia including, but not limited to, humans, non-human primates (e.g., chimpanzees, other apes, and monkey species), farm animals (e.g., cattle, horses, sheep, goats, and swine), domestic animals (e.g., rabbits, dogs, and cats), or laboratory animals including rodents (e.g., rats, mice, and guinea pigs). Examples of non-mammals include, but are not limited to, birds, and the like. The term “subject” does not denote a particular age or sex. In one specific embodiment, a subject is a mammal, preferably a human. The subject may have or suffer from, or be suspected of having or suffering from, a disease, condition, or disorder.

In some embodiments, the subject may have or suffer from, or be suspected of having or suffering from, an infection by a pathogen. “Pathogen” means an organism that can produce disease in a subject. Examples of pathogens include, without limitation, viruses, bacteria, fungi, and parasites. Accordingly, the methods and systems described herein may be used to detect an antigen that originates from a pathogen or an antibody produced in response to the subject's infection by a pathogen. The Examples demonstrate detection of antigens that originate from a pathogenic source, e.g., SARS-CoV-2, but the antigens may originate from non-pathogenic sources, including without limitation self-antigens that originate from the subject.

In particular embodiments, the pathogen may be associated with a pulmonary or respiratory disease. Exemplary pathogens include enveloped, single-stranded positive-sense RNA virus of the family Coronaviridae and optionally of the genus Betacoronavirus. Exemplary viruses of the genus include SARS-CoV-2, SARS-CoV, and MERS-CoV. Examples demonstrate the detection of antigens originated from SARS-CoV-2 as well as antigens associated with influenza A, enterovirus, H1N1 (swine flu), and pneumonia. Antigens or antibodies from other sources may also be detected.

In some embodiments, the virus infecting the subject is SARS-CoV-2. SARS-CoV-2's RNA genome encodes many viral proteins, including four structural and 16 nonstructural proteins. An antibody to these viral proteins may be tethered to the microcantilever and binding of the viral protein with the antibody may be detected. In some embodiments, an antibody to a receptor binding domain (RBD) protein, such S1 or N, may be tethered to the microcantilever.

The advantages of the systems and devices of this disclosure are multifold and include, for example, the benefit of shortening the time between sample collection and analysis, thereby identifying subjects having particular test results and, if appropriate, providing medical treatment.

In some embodiments, the methods further comprise treating the subject. The terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration.

Antibody-functionalized microcantilevers comprise a microcantilever, an antibody configured to bind a target antigen or an antigen configured to bind a target antibody, and a tether covalently tethering the antibody or the antigen to the microcantilever.

The antibody-functionalized microcantilevers comprise an effective surface density of tethered antibodies or tethered antigens. As used herein, “effective surface density” means an amount of tethered antibodies or tethered antigens that when in contact with sample comprising the target antigen or antibody is capable of inducing a detectable response. Antibody-functionalized microcantilevers may be prepared by contacting tether-functionalized microcantilevers with a precursor solution comprising antibodies or antigens for the desired target such that the antibodies or antigens can covalently bond to the tethers. In some embodiments, the precursor solution comprises between 10 μg/ml-100 μg/ml of the antibody or antigen to be bound.

In some embodiments, the effective surface density is capable of inducing a detectable response when the sample comprises a clinically relevant concentration of target antigen or antibody. As used herein, “clinically relevant concentration” of target antigen or antibody is a concentration of the target antigen or antibody indicative of a disease, disorder, or condition, such as an infection by SARS-CoV-2 or another pathogen.

In some embodiments, the effective surface density is capable of inducing a detectable response when the sample comprises at least 1 μg/ml, 100 ng/ml, 10 ng/ml, or at least 1 ng/ml of target antigen or antibody. In some embodiments, the effective surface density is capable of inducing a detectable response when the sample comprises less than 1 ng/ml of target antigen or antibody.

The microcantilever is composed of one or more materials that can respond to antibody-antigen binding and covalently bond with a tether. Suitably, the microcantilever may be composed of silicon coated with gold as in the Examples but other materials may also be used.

The microcantilever is also dimensioned to allow for a response in response to antibody-antigen binding. The microcantilever may also be dimensioned to have length from about 250 μm to 600 μm, and width of 50 μm to 100 μm with thickness of 1 μm to 2 μm. The Examples demonstrated the use of microcantilevers having a length of 500 μm, width of 80 μm in width, and thickness of 1.2 μm but other lengths, widths, and thickness may be used.

The cantilevers may be tipped or tipless depending on detection methodology. The Examples demonstrated the use of tipless cantilevers where detection was performed optically. However, tipped cantilevers may be used where detection is performed electrically. Example 1 demonstrates rapid and sensitive detection of SARS-CoV-2 antigen proteins in clinical samples using microcantilever platform through optical detection method for nanomechanical sensing. Example 2 demonstrated electrical detection based non-invasive method is disclosed for rapid detection of antigenic proteins through exhaled breath of the patients with a high degree of sensitivity and selectivity. The electronics readout of MOSFET-embedded microcantilever sensors was used to detect various respiratory pathogen deflection, including SARS-Cov-2.

The tethers covalently tether the antibody or antigen to the microcantilever. The tether may be a heterobifunctional molecule with a spacer between immobilization and conjugation functional moieties. The Examples demonstrate the use of a thiocarboxylic acid, where the thiol group is used to immobilize the tether to the microcantilever via gold-thiol bonding, the carboxylic acid moiety is used to conjugate the antibody to the tether, and an alkyl chain provides a spacing and conformation flexibility between the antibody and microcantilever. The immobilization group, conjugation group, and spacer may be appropriately selected based on the material of the microcantilever and antibody or antigen to be conjugated.

Conjugation is a chemical strategy for forming stable covalent linked between molecules, where one is a biomolecule such as the antibodies and antigens used herein. Conjugation chemistries often use carboxyl, amine, thiol, or bio-orthogonal groups that target functional groups that are not native in biomolecules. The Examples demonstrate the use of EDC-NHS chemistry, but other conjugation chemistry may also be used and are generally known in the art.

Another aspect of the invention provides for systems for the detection of a target antigen or target antibody in a sample. The system comprises any of the nanomechanical sensors described herein and a detector for detecting a response of the nanomechanical sensor to the sample and generating a signal indicative of the response of the nanomechanical sensor to the sample.

In some embodiments, the detector can detect a bending moment response. In some embodiments, the bending moment is detected by optically detecting a bending moment. In other embodiments, the bending moment is detected by electrically detecting a bending moment.

In some embodiments, the system is configured for multiplexed detection. Suitably, the system may comprise two or more antibody-functionalized cantilevers where different antibodies, antigens, or combinations or antibodies and antigens are tethered to different cantilevers. Where the system comprises two or more antibody-functionalize cantilevers, the detector may be configured to individually detect the response of each cantilever and generate a signal indicative of the response of each sensor.

In some embodiments, the different antibody-functionalized cantilevers are configured to detect antibodies or antigens that originate from the same source. In some embodiments, the system is configured to detect two or more antibodies, antigens, or a combination thereof that originate from the same pathogen. For example, the system may be configured to detect both the SARS-CoV-2 Spike (S1) protein and the SARS-CoV-2 Nucleocapsid (N) protein.

In other embodiments, the different antibody-functionalized cantilevers are configured to detect antibodies or antigens that originate from different sources. In some embodiments, the system is configured to detect two or more antibodies, antigens, or a combination thereof that originate from the different pathogens, including different species or different variants, or a pathogen and the subject for a control.

In some embodiments, the system further comprises a computing platform having a communication interface that receives one or more signals from the detector, and a computer in communication with the communication interface, wherein the computer comprises a computer processor and a computer readable medium comprising machine-executable code that, upon execution by the computer processor, implements a method for determining the response of the one or more nanomechanical sensors to the sample.

The computational platform is capable of characterizing and determining the response of one or more nanomechanical sensors to the sample. The computational platform may generally include various input/output (I/O) modules, one or more processing units, a memory, and a communication network.

In some implementations, the computational platform may be any general-purpose computing system or device, such as a personal computer, workstation, cellular phone, smartphone, laptop, tablet, or the like. In this regard, the computational platform may be a system designed to integrate a variety of software, hardware, capabilities, and functionalities. Alternatively, and by way of configurations and programming, the computational platform may be a special-purpose system or device.

The computational platform may operate autonomously or semi-autonomously based on user input, feedback, or instructions. In some implementations, the computational platform may operate as part of, or in collaboration with, various computers, systems, devices, machines, mainframes, networks, and servers. For instance, the computational platform may communicate with one or more servers or databases, by way of a wired or wireless connection.

The I/O modules of the computational platform may include various input elements, such as a mouse, keyboard, touchpad, touchscreen, buttons, microphone, and the like, for receiving various selections and operational instructions from a user. The I/O modules may also include various drives and receptacles, such as flash-drives, USB drives, CD/DVD drives, and other computer-readable medium receptacles, for receiving various data and information. To this end, I/O modules may also include a number of communication ports and modules capable of providing communication via Ethernet, Bluetooth, or Wi-Fi, to exchange data and information with various external computers, systems, devices, machines, mainframes, servers, networks, and the like. In addition, the I/O modules may also include various output elements, such as displays, screens, speakers, LCDs, and others.

The processing unit(s) may include any suitable hardware and components designed or capable of carrying out a variety of processing tasks, including steps implementing the present framework for response detection. To do so, the processing unit(s) may access or receive one or more signals generated by the detector. The signals may be stored or tabulated in the memory, in the storage server(s), in the database(s), or elsewhere. In addition, such information may be provided by a user via the I/O modules or selected based on user input.

In some configurations, the processing unit(s) may include a programmable processor or combination of programmable processors, such as central processing units (CPUs), graphics processing units (GPUs), and the like. In some implementations, the processing unit(s) may be configured to execute instructions stored in a non-transitory computer readable-media of the memory. The non-transitory computer-readable media may be included in the memory, it may be appreciated that instructions executable by the processing unit(s) may be additionally, or alternatively, stored in another data storage location having non-transitory computer-readable media.

In some configurations, the processing unit(s) may include one or more dedicated processing units or modules configured (e.g., hardwired, or pre-programmed) to carry out steps, in accordance with aspects of the present disclosure. Each solver module may be configured to perform a specific set of processing steps, or carry out a specific computation, and provide specific results

The processing unit(s) may also be configured to generate a report and provide it via the I/O modules. The report may be in any form and provide various information. For instance, the report may include various numerical values, text, graphs, maps, images, illustrations, and other renderings of information and data. In particular, the report may provide various information or properties generated by the processing unit(s) from the detector generated signals. The report may also include various metrics or indices for determining whether a target antigen or antibody is present in a sample. To this end, the report may be provided to a user, or directed via the communication network to an assembly line or various hardware, computers, or machines therein.

In some embodiments, the systems and devices of this disclosure can analyze samples at the point-of-need rather than in a laboratory. As used herein, the term “point of need” or “point of care” can be defined to mean a location on or near a site of patient care where medical or medically related services such as medical testing and/or treatment are provided, including but not limited to hospitals, emergency departments, intensive care units, primary care setting, medical centers, patient homes, physician offices, pharmacies, or sites of an emergency.

In summary, the Examples demonstrate microcantilever-based nanomechanical detection of SARS-CoV-2 in patient nasopharyngeal swab and breath samples with high degree of sensitivity, specificity, and selectivity in just a few minutes (“collection to signal” in less than 10 mins). The patient sample diagnostic selectivity was optimized for SARS-CoV-2 spike (S1) and nucleocapsid (N) proteins specific detection by microcantilever sensor platform. The sensitivity of microcantilever sensors is such to detect SARS-CoV-2 S1 and N proteins at concentrations as low as 1 ng/ml (33 pM), within 2-3 minutes of probe-target interaction. Furthermore, microcantilevers exhibit no measurable cross-reactivity with non-specific N/S1 proteins in different and varied combinations showing a high degree of specificity and patient sample data provide favorable selectivity. These results collectively demonstrate efficacy for highly sensitive, multiplexed, and rapid detection of antigens associated with SARS-CoV-2, responsible for the worldwide pandemic of COVID-19. The Examples also show that the spike protein (RBD) from mutant variant of SARS-CoV-2 (B.1.17) appears to exhibit lower binding affinity to its corresponding antibody derived from the wild-type strain. Accordingly, nanomechanical signal transduction allows for rapid and real-time assays based on specific binding on microcantilever arrays.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus≤10% of the particular term and “substantially” and “significantly” will mean plus or minus>10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1

### Materials

SARS-CoV-2 Chimeric monoclonal antibody for Spike (S1) protein (40150-D003), SARS-CoV-2 Chimeric monoclonal antibody for Nucleocapsid (N) protein (40588-R0004), SARS-CoV-2 Spike (RBD) protein (40150-V08B2), and nucleocapsid (N) protein (40588-V08B) were all purchased from Sino Biological Inc. Phosphate Buffer Saline (PBS), PBS-Tween 20 sachets, Bovine Serum Albumin (BSA), and 11-Mercaptoundecanoic acid (MUA) were purchased from Millipore-Sigma. 1-Ethyl-3-(3-dimethylaminopropyl) carbodiimide and Sulfo-NHS were received from ThermoFisher Scientific. Nasopharyngeal swab specimens were collected from individuals presenting to Northwestern Memorial Hospital, Chicago. The spike protein (S1-RBD) from mutant UK variant B.1.1.7 was also purchased from Sino Biological Inc (40591-V08H12). The set-up used for the deflection experiments was Bruker Bioscope Resolve liquid imaging system. The tipless silicon cantilevers used in these experiments were acquired from Nanoworld Incorporation.

### Experimental Methods

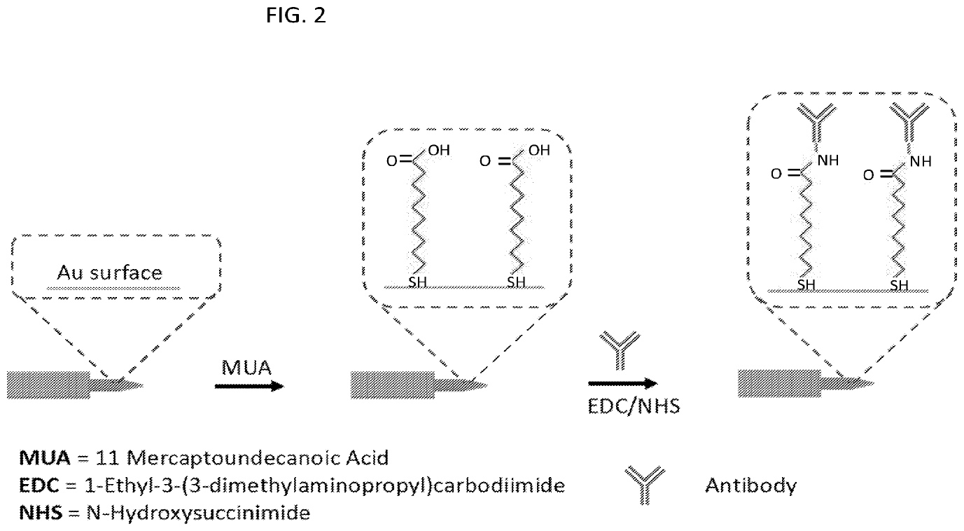

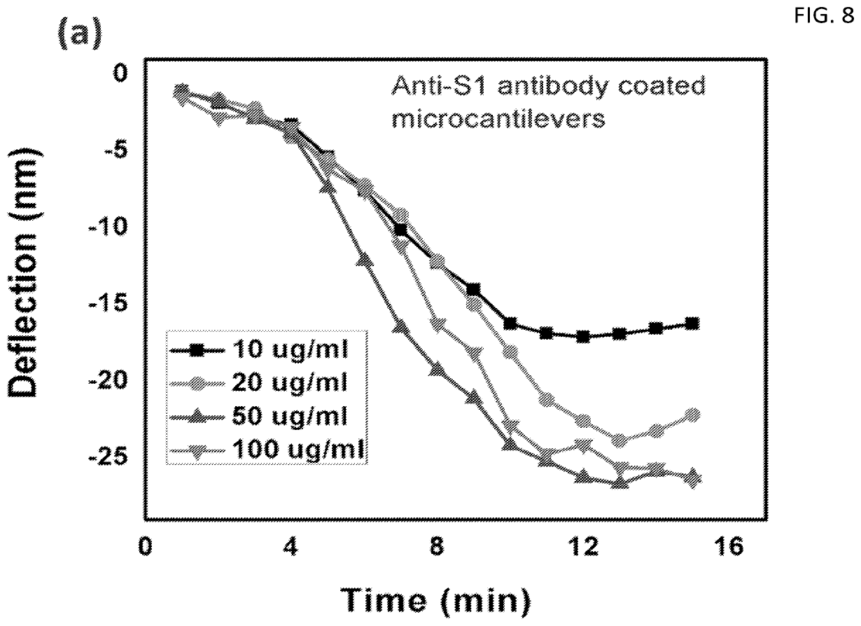

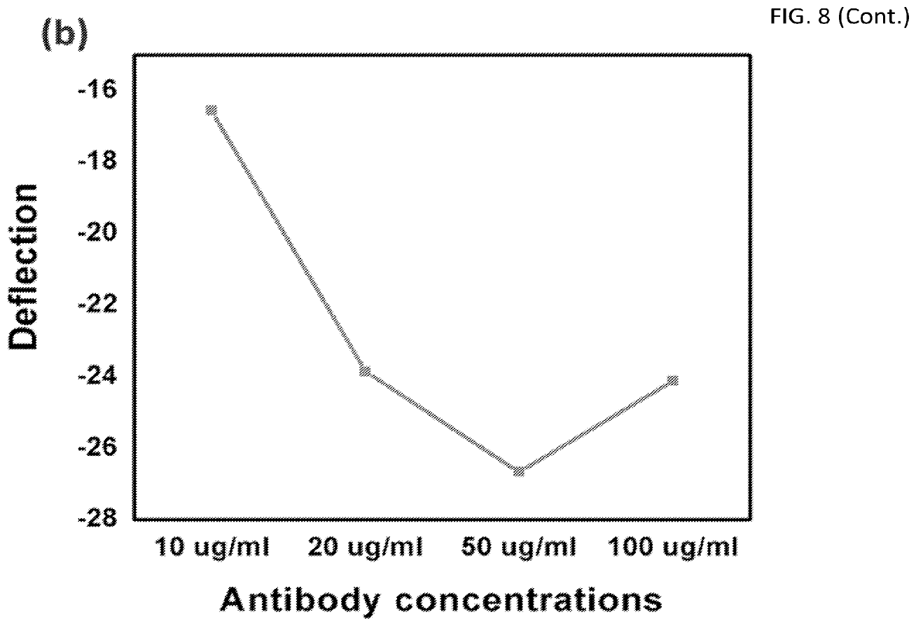

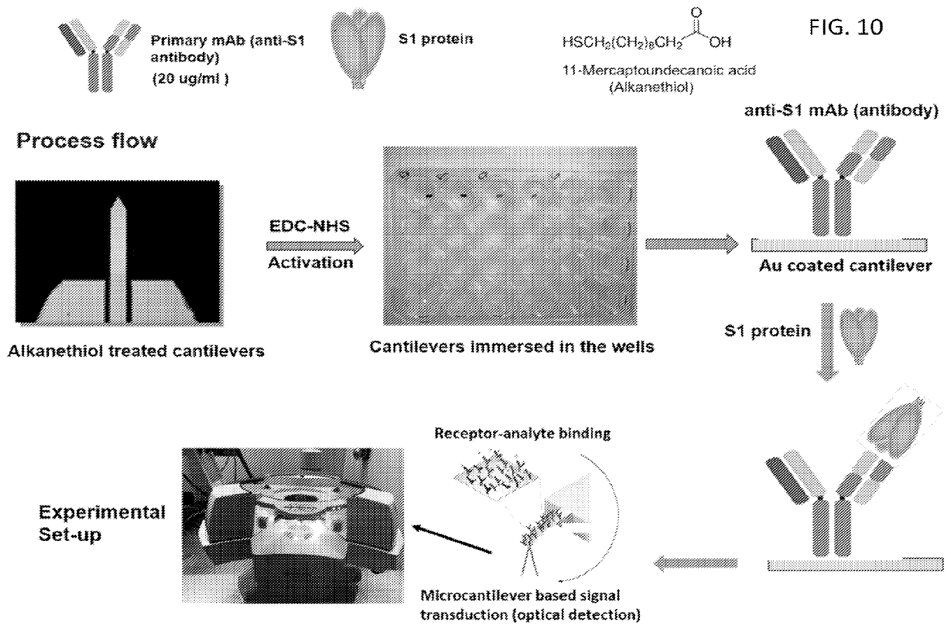

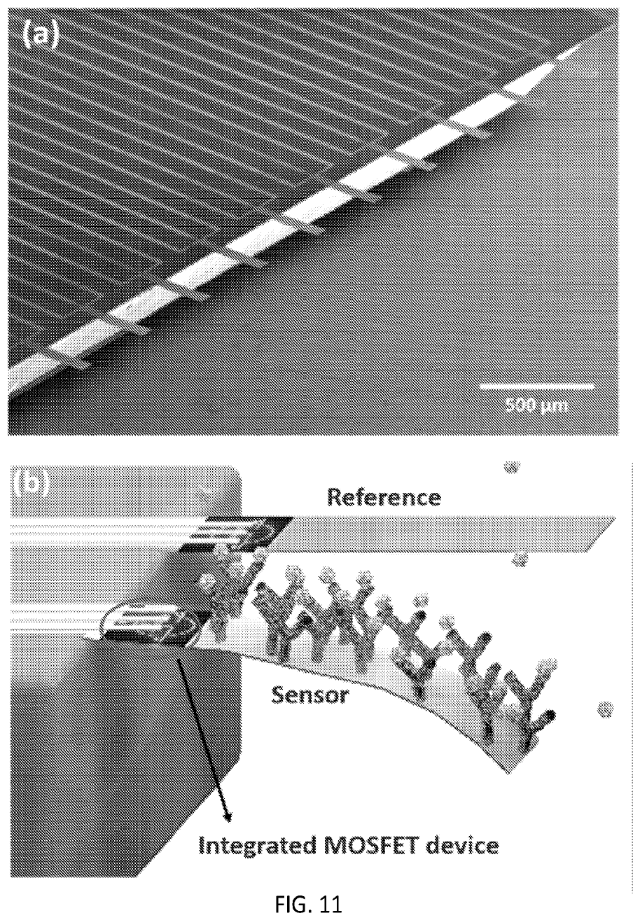

Preparation and Functionalization of the Microcantilever Surface: Prior to use, the gold coated cantilevers were plasma cleaned prior to antibody immobilization to remove any organic residues. The anti-SARS-CoV-2 antibodies (for S1 and N proteins) were covalently attached onto the gold coated cantilever surface through EDC-NHS chemistry inside a glass plate containing tiny wells (See FIG. 10). First, the cantilevers were immersed in 10 mM solution of 11-Mercaptoundecanoic acid (MUA) prepared in Ethanol to graft Carboxylic groups on the surface followed by rinsing multiple times through DI water. The activation of these carboxylic groups was carried out by putting the cantilevers for incubation in a 100 μl solution mixture of 5 mM Carbodiimide EDC and 5 mM Sulfo-NHS in DI water. After rinsing with DI water, these functionalized microcantilevers were incubated overnight in 20 μg/ml of antibody solutions (separately for S and N proteins) prepared in PBS and 0.05% BSA (pH=7.4) to facilitate the covalent immobilization (FIG. 2). We first performed multiple experiments to optimize the antibody coverage on the cantilever surface and chose 20 μg/ml as optimum concentration for the deflection measurements (see FIG. 8). Microcantilevers were then rinsed with PBS-tween-20 solution, dried and then fixed in the AFM sample holder.

Experimental Detection Method: Specific biomolecular binding results in lateral stress that imposes a bending moment on the free end of the microcantilever. The microcantilever bends upwards or downwards in response to a tensile or compressive surface stress, respectively. This deflection can be monitored real-time with exquisite sensitivity, similar to AFM in operation. The receptor-coated microcantilever is typically positioned next to a passivated reference cantilever for differential signal of deflection as well as added redundancy, duplication ad multiplexing for improved specificity. The sensitivity in these measurements is governed by ability to measure the slightest deflections quantitatively and reproducibly.

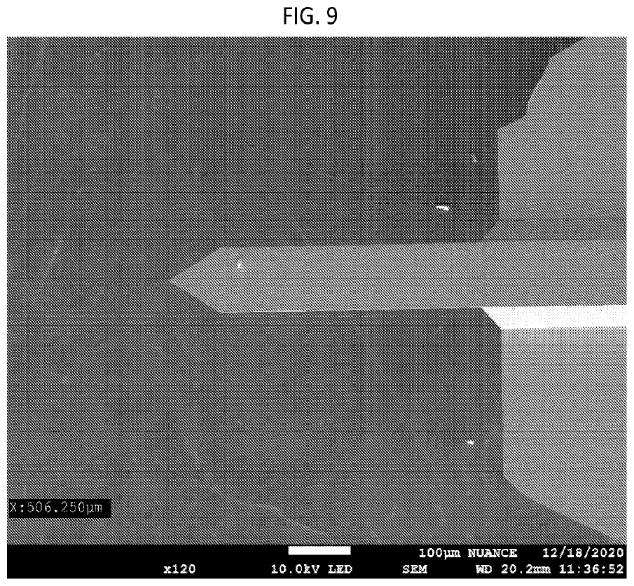

The tipless gold coated silicon cantilevers used in these experiments (500 μm long, 100 μm wide and 1 μm in thickness) were acquired from Nanoworld Incorporation (See FIG. 9). The cantilevers and experimental conditions used in each measurement were similar. The absolute deflection at the free end of each cantilever (Δz) was measured using an optical detection system. The deflection signal results into a differential surface stress Δσ, using Stoney's equation,

$$\\ Δ σ = 1 / 4 \cdot {( t / L )}^{2} \cdot E / ( 1 - v ) \cdot Δ z$$

where L is the effective length of the cantilever, t is the thickness, and E/(1-ν) is the ratio between the Young's modulus E (130 GPa) and Poisson ratio ν of Si (0.28). The force constant of these cantilevers was around 0.03 N/m. Because a cantilever deflection depends on geometry, all the cantilevers used in these measurements were exactly similar in their geometry. The cantilever positive (upward) or negative (downward) bending due to change in surface stress as a result of antibody-antigen interaction is directly related to the quantitative measurements for the bindings. The deflection experiments were performed inside a microfluidic reaction chamber sized approximately 2 mm in diameter. The biofunctionalized microcantilevers was brought in close proximity to a microfluidics chamber through stepper motor which contains antigen solution (3 μl approx.) The deflection experiments were performed for different concentrations of SARS-CoV-2 spike (S1) and nucleocapsid proteins (N), in addition to actual patient samples. As the cantilever starts bending due to surface stress, vertical deflection of the cantilever was measured.

### Detection of SARS-CoV-2 Spike (S1) Protein.

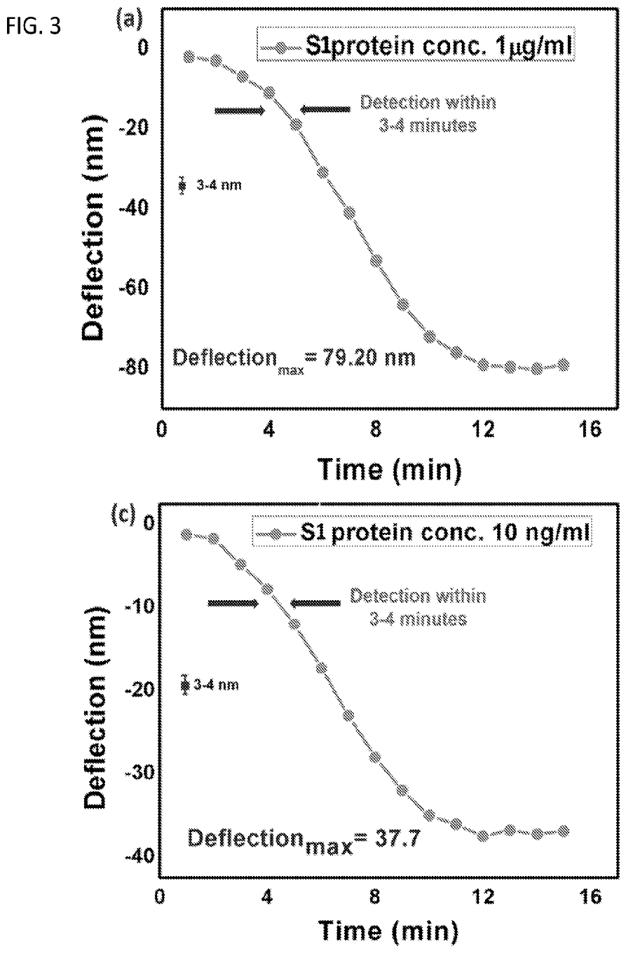

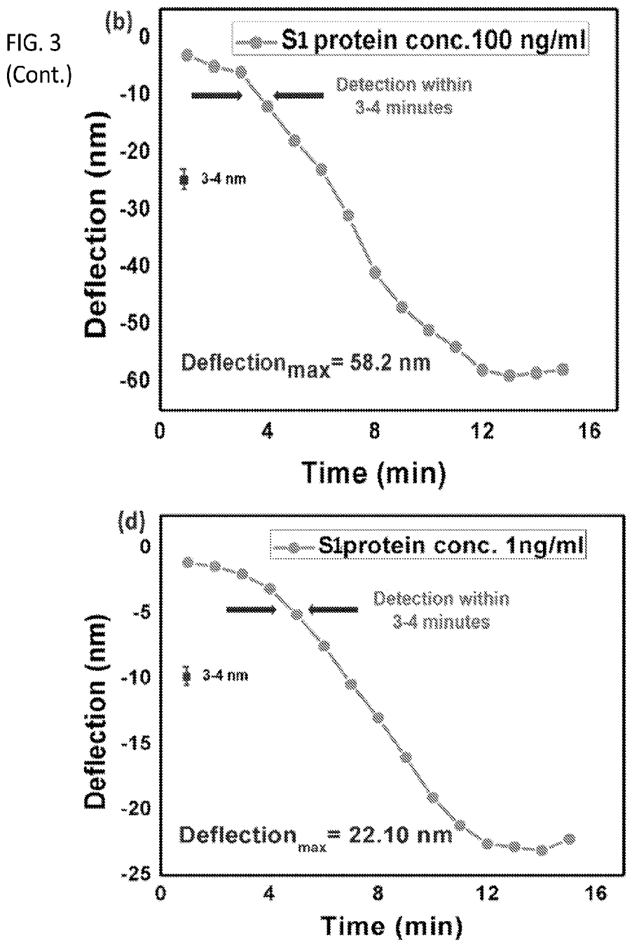

The micro cantilever based optical detection system was employed to detect SARS-CoV-2 S1 proteins. Apart from being a major transmembrane structural protein, spike protein is also highly immunogenic and possesses diverse amino acid sequences among the corona virus family making it an important target to develop a specific diagnostic platform for SARS-CoV-2 Microcantilevers biofunctionalized with anti-S1 protein antibody (20 μg/ml) were used to quantify the detection signal using different concentrations of S1 protein (1 μg/ml, 100 ng/ml, 10 ng/ml, and 1.0 ng/ml, respectively). The real-time deflection signal was observed for 15 minutes, as demonstrated in FIG. 3. We found that deflection signal was observed within 3-4 minutes in presence of viral antigen due to specific and selective interaction between the antibody and viral antigen.

The deflection signal (Δz) exhibited descending trends with decreasing antigen concentrations, i.e., 1 μg/ml (79.20+/−3-4 nm), 100 ng/ml (58.20+/−3-4 nm), 10 ng/ml (37.70+/−3-4 nm), and 1 ng/ml (22.10+/−3-4 nm), respectively, demonstrating highly specific binding between anti-S1 antibody and S1 protein. As illustrated in the FIG. 3 (a), the cantilever with highest S1 concentration (1 μg/ml) showed a maximum bending signal (79.20+/−3-4 nm) whereas cantilever with the lowest antigen concentration showed the minimum response. These results can be attributed to the biomolecular interaction between the antibody and its corresponding target antigen resulting in an increased compressive stress on the cantilever surface, generating a large deflection signal. The observed signal was reproducible with in +3-4 nm of variation for a given concentration after running series of experiments.

### Detection of SARS-CoV-2 Nucleocapsid (N) Protein.

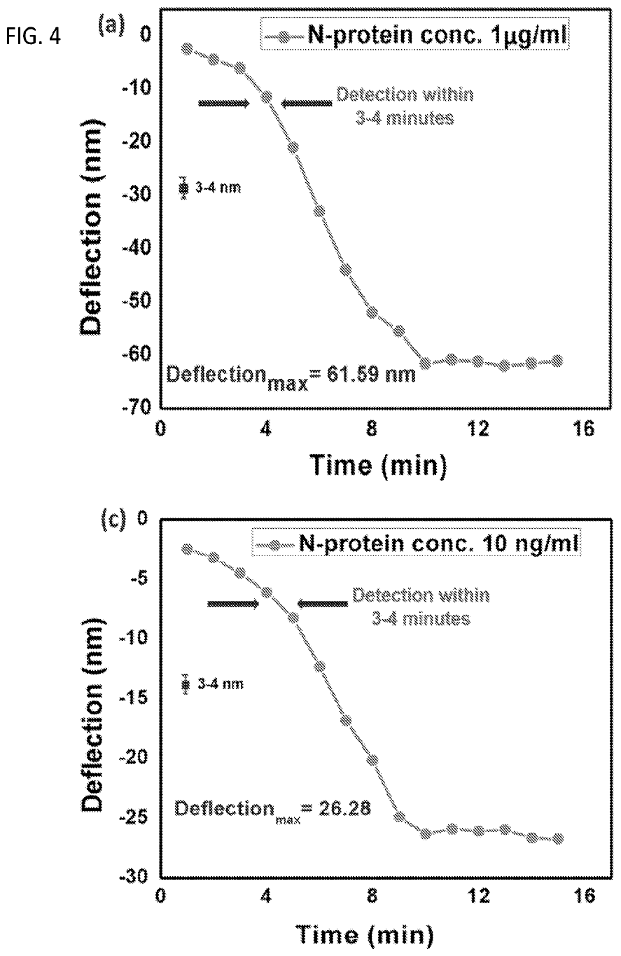

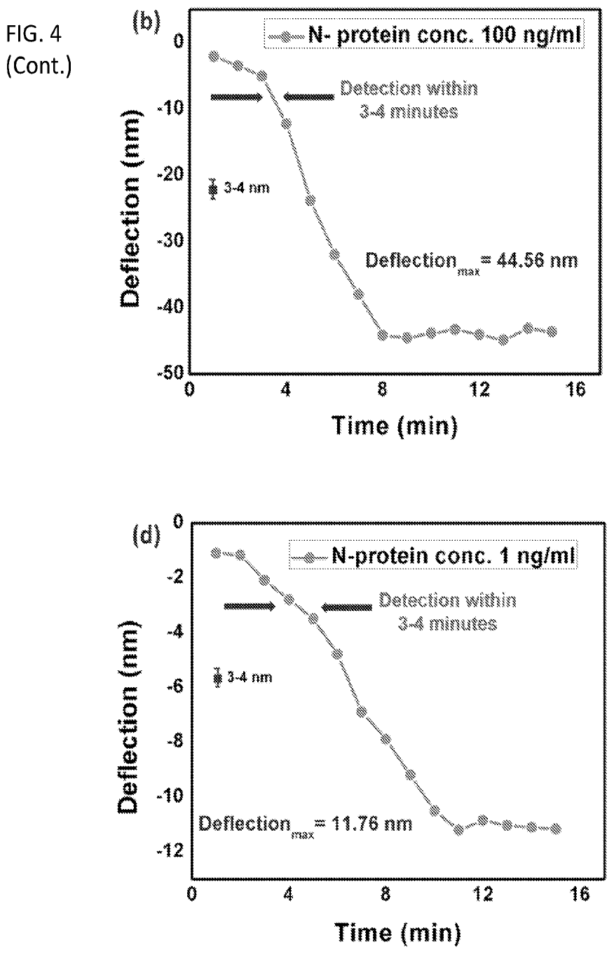

The cantilever response towards the SARS-CoV-2 Nucleocapsid (N) protein was evaluated. It is one of the 5 major non-structural proteins of coronavirus which is also highly immunogenic and has more stable and conserved amino acid sequences among the coronaviruses. This led us to study N protein mediated cantilever deflection and a comparison with S1 protein. Microcantilevers biofunctionalized with anti-N protein antibody (20 μg/ml) were used for the deflection measurements (FIG. 4).

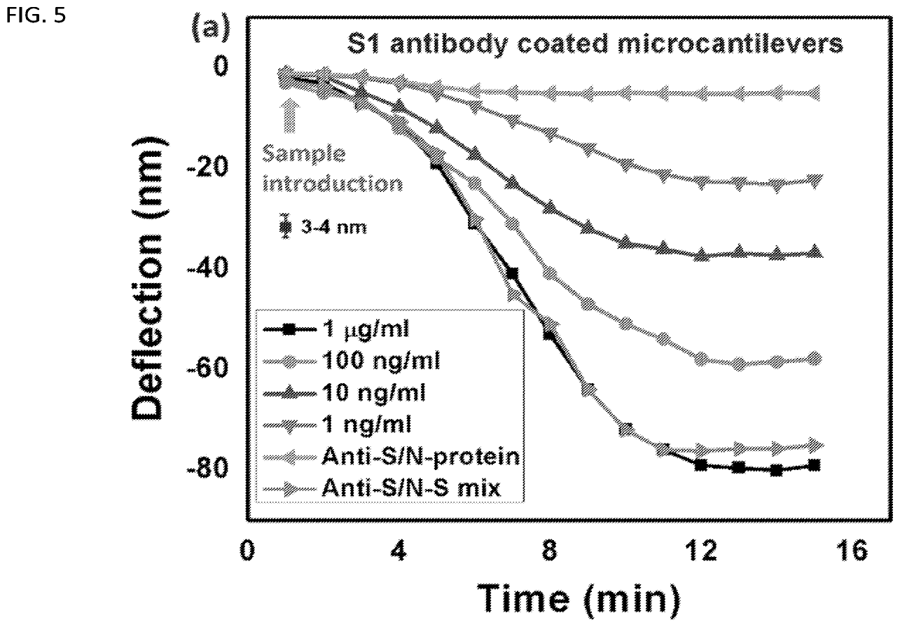

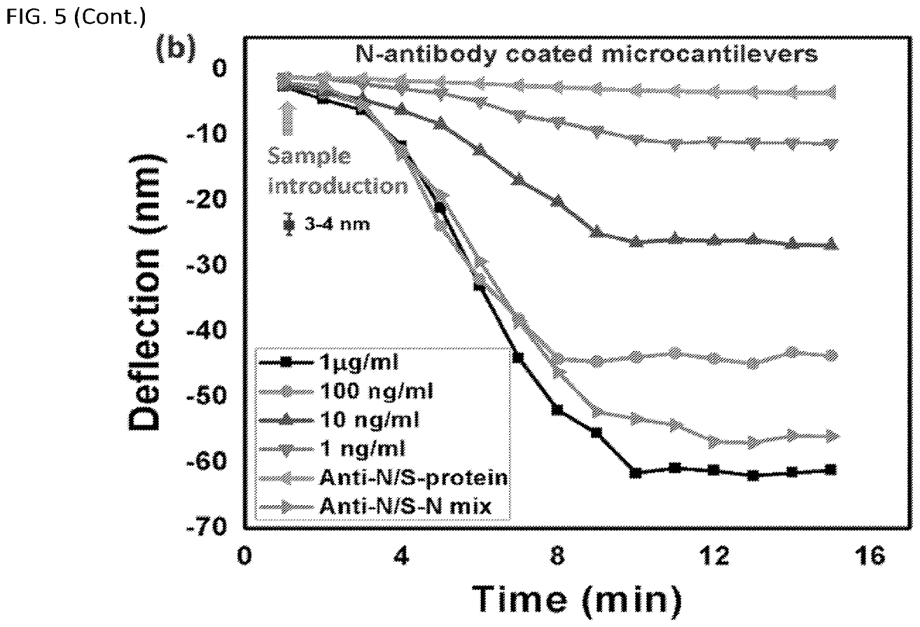

As shown in FIG. 4, the maximum cantilever deflection (61.59+/−3-4 nm) was measured for 1 μg/ml of N-protein concentration, whereas the minimum cantilever deflection (11.76+/−3-4 nm) was exhibited by the lowest antigen concentration (1 ng/ml). FIGS. 4 and 5 show that the observed values for cantilever deflection in the case of S1-protein was larger in comparison with N protein for all the concentrations used which can be attributed to the difference in the magnitude of the surface stress generated in both cases.

### Specificity in the Detection.

To establish the specificity and to greatly increase the efficacy of our detection method to specific markers, ross-validation experiments to check the cross-reactivity with non-specific proteins were performed. First, the cantilever coated with anti-S1 antibody was allowed to react with SARS-CoV-2 N protein (1 μg/ml) and data was recorded. FIG. 5 (a) (left-pointing arrow) shows that the cantilever device did not show any significant deflection signal (approximately 3-4 nm) in the absence of any antibody-antigen interaction on its surface after the introduction of N protein. Furthermore, another cantilever coated with anti-S1 antibody was allowed to interact with a mixture of N and S1 protein solution (Anti-S/N-S mix), taking 1 μg/ml concentration for each protein. As shown in FIG. 5 (right-pointing arrow), the deflection profile is similar (76+/−3-4 nm) to the detection of S1 protein alone (square) indicating a specific anti-S1 antibody-S1 protein interaction. Similar experiments were performed for anti-N antibody coated cantilevers (FIG. 5) which were then interacted with S1 protein (left-pointing arrow) and a mixture of N and S1 proteins (anti-N/S-N mix) in separate experiments. These cantilevers also demonstrated the specific reaction towards the N protein with a maximum bending signal and a negligible deflection with the non-specific S1 protein. The experiments conducted here ascertain that the sensor platform is not only sensitive, but highly specific for SARS-CoV-2 spike and nucleocapsid protein detection. Table 1 displays the deflection values for S1 and N protein from the optical measurements.

TABLE 1
Probe-target biomolecular interactions in terms
of nanomechanical responses of microcantilevers
for different concentrations of S1 and N proteins

| | S1- protein | N- protein |
|---|---|---|
| | deflection | deflection |
| | (nm) +/− | (nm)+/− |
| Target concentration | 3-4 nm | 3-4 nm |
| 1 μg/ml (3.3 × 10⁴ pM (S1)/2.1 × | 79.20 | 61.59 |
| 10⁴ pM (N) | | |
| 100 ng/ml (3.3 × 10³ pM (S1)/2.1 × | 58.23 | 44.56 |
| 10³ pM (N) | | |
| 10 ng/ml (3.3 × 10² pM (S1)/2.1 × | 37.72 | 26.28 |
| 10² pM (N) | | |
| 1 ng/ml (33.0 pM (S1)/21.0 pM (N) | 22.10 | 11.76 |

After successfully demonstrating the specificity in SARS-CoV-2 S1 and N protein detection, the cantilever sensor was then employed for a control experiment also. Here, the cantilever was not conjugated with any antibody (anti-S1/anti-N) which was further allowed to interact with its corresponding antigen protein. In the absence of any antibody-antigen interaction, there was no significant bending of the microcantilever. The control experiment indicates that an antibody conjugation is necessary on the cantilever surface for a specific binding with its complementary antigen to trigger an antibody-antigen interaction.

### SARS-CoV-2 Detection in Clinical Samples From COVID-19 Patients: Selectivity of Microcantilever Assay.

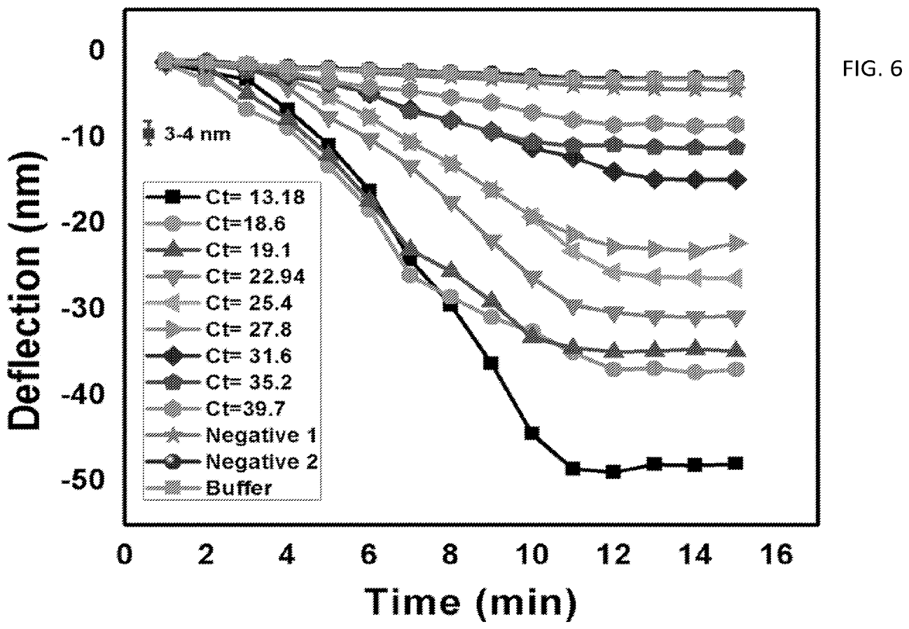

Having successfully detected S1 and N proteins in isolation, the selectivity of our microcantilever system for detecting SARS-CoV-2 in patient specimens was evaluated. As per standard protocol, the nasopharyngeal swabs were stored in viral transport medium (VTM) and transported to the pathology laboratory. Prior to testing, the samples underwent a viral heat inactivation procedure by incubating them in a water bath at 65° C. for 30 minutes. The positive samples used for the study had a spectrum of Ct values consistent with high (low Ct value) and low (high Ct value) viral load. The information regarding patient sample collection and processing is further described below. The deflection measurements were carried out with respect to the different Ct values from 9 different patient samples. As seen in FIG. 6, the positive sample with the highest Ct value (39.7) yielded the lowest deflection (8.46+/−3-4 nm) whereas maximum deflection (48.91+/−3-4 nm) was exhibited for the patient sample having the lowest Ct value (13.18). This is consistent with the fact that samples with the high Ct values generally have low concentrations of N protein (≈0.71 ng/ml for the highest Ct value (39.71)-calculated from the standard N protein data) present in the solution to bind on the cantilever surface as opposed to the sample with the low Ct value. More and high affinity binding events result in increase in the resultant stress on the cantilever surface and thus more deflection.

In addition, the deflection signal from samples from subjects without SARS-CoV-2 infection (negative samples) was evaluated. As shown in FIG. 6 (ball and star lines), there was no significant deflection for the samples taken from non-COVID individuals (negative). Negative sample with buffer also failed to yield any measurable deflection (square line). This implies that there is little or no nucleocapsid protein present in the sample to bind to its target antibody. This demonstrates that the methodology is capable of distinguishing SARS-CoV-2 antigen proteins in patient samples from the general array of proteins found in specimens obtained from healthy persons. The data demonstrate potential practical and rapid (<10 minutes from swab to signal) detection of SARS-CoV-2 antigens in transport medium of the patient sample with high sensitivity, specificity, and selectivity.

The different Ct values and the corresponding cantilever deflection from SARS-CoV-2 positive and negative individuals are given below in Table 2.

TABLE 2
Deflection signal with respect to the Ct values
from different nasopharyngeal swab specimens

| | | Deflection (nm)+/− |
|---|---|---|
| Patients | Ct values | 3-4 nm |
| Positive 1 | 13.18 | 48.91 |
| Positive 2 | 18.60 | 36.95 |
| Positive 3 | 19.12 | 33.18 |
| Positive 4 | 22.94 | 30.72 |
| Positive 5 | 25.41 | 25.62 |
| Positive 6 | 27.85 | 22.86 |
| Positive 7 | 31.62 | 13.89 |
| Positive 8 | 35.20 | 11.05 |
| Positive 9 | 39.71 | 8.46 |
| Negative 1 | 0 | 4.13 |
| Negative 2 | 0 | 3.02 |
| Buffer | 0 | 3.14 |

Binding Study for Mutant Strain from U.K. Variant (B.1.1.7): Potential Variation in Specificity

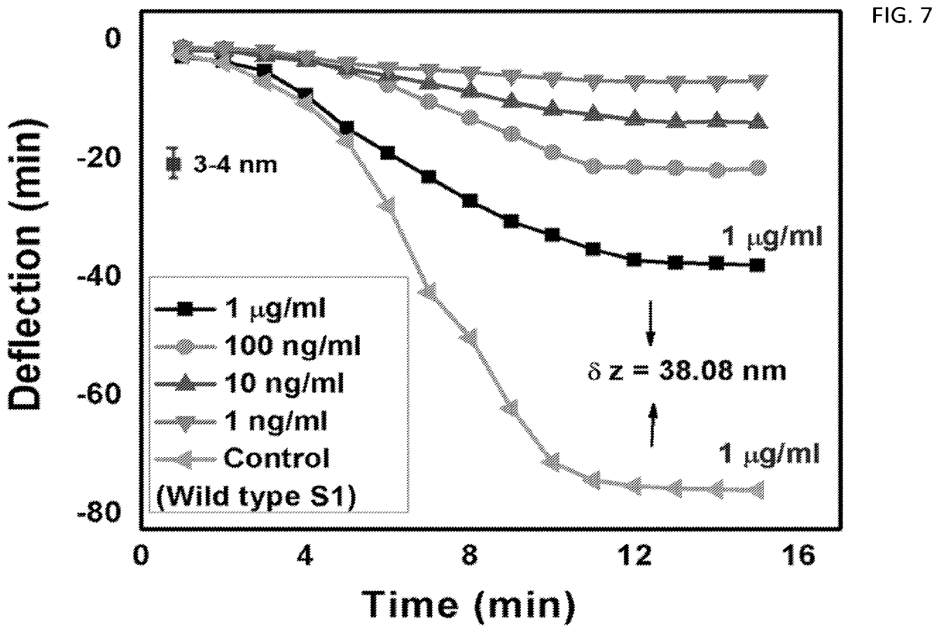

Since new SARS-CoV-2 variants are emerging rapidly, following specific detection of SARS-CoV-2 in patient samples, we further extended the experiments to study the binding affinity of spike protein (S1) from mutant U.K. variant (B.1.1.7) with wild type antibodies from the original strain (wild type). This variant carries different mutations in its RBD region (H69del, V70del, Y144del, N501Y, A570D, D614G, P681H). It is of particularly interest whether the mutations in receptor binding domain (RBD) of spike protein affect the binding with its complementary antibody from the original strain and whether it shows the same sensitivity and specificity as their wild type counterpart. This is obviously crucial for designing therapeutics and vaccines as well as assessing the antigenic implications of viral evolution, among others. Four different concentrations of S1 protein (1 μg/ml, 100 ng/ml, 10 ng/ml, and 1 ng/ml) were used to make a comparison with our data from the original strain. Spike (RBD) protein from the original strain (1 μg/ml) was used as a control. Cantilever functionalization and antibody grafting were carried out in a similar fashion as mentioned earlier in this work. As seen in FIG. 7, there is a contrast between wild type and mutant strain in the cantilever deflections for all the concentrations tested. The maximum deflection obtained for the highest concentration (1 μg/ml) was 75.61+/−3-4 nm for the wild-type strain whereas for mutant, it was measured 37.53+/−3-4 nm. Similarly, for the lowest concentration (1 ng/ml), the mutant strain exhibited a deflection of 6.85+/−3-4 nm in contrast to the wild type (22.10+/−3-4 nm). Table 3 shows the deflection data from both wild type and the mutant strain.

TABLE 3
Microcantilever deflection data for both wild type and
mutant UK variant with respect to the concentration

| | | |
|---|---|---|
| | Deflection (wild type | Deflection (mutant UK |
| Concentrations | strain) +/− 3-4 nm | strain) +/− 3-4 nm |

| | | | | |
|---|---|---|---|---|
| 1 | μg/ml | 75.61 nm (control) | 37.53 | nm |
| 100 | ng/ml | 58.23 nm | 21.52 | nm |
| 10 | ng/ml | 37.72 nm | 13.81 | nm |
| 1 | ng/ml | 22.10 nm | 6.85 | nm |

These experimental findings demonstrate that the mutations in the RBD region of spike protein have an impact on its binding affinity with its wild type receptor molecule (antibody) on the cantilever surface and thus the specificity.

### Microcantilever Based Sensing Phenomenon

The Examples demonstrate specific and selective detection of both N and S1 (RBD) proteins of the virus at a concentration of less than 1 ng/ml (33 pM). A site-directed antibody immobilization technique was adopted to attach antibodies for S1 and N proteins on gold coated microcantilever using EDC-NHS chemistry. This method facilitates a specific bio-molecular binding between ligands and receptors on the surface of a microcantilever beam resulting in physical bending of the beam by some tens of nanometers. The Examples demonstrate that the assay exhibited an excellent sensitivity and specificity based on cantilever deflection measurements. The specific-biomolecular interaction results in lateral stress that induces a torque on the microcantilever resulting in deflection of the cantilever beam. This reflects the interplay between the strain energy increase of the cantilever and the free energy reduction of the interaction. It provides a unique approach for investigating the connection between the nanomechanics and the chemistry of antibody-antigen interaction at very low concentration. This qualifies the technology as a rapid method to detect the SARS-CoV-2 and other pathogens that reveals disease risk and its progression. This approach provides a label free signature for specific binding events and is readily amenable to not only massively parallel and multiplexed sensing but fully compatible microfluidics with all the advantages of the commercially successful microelectronics platform.

### Antibody Coverage on the Microcantilever Surface

Prior to deflection measurements, microcantilevers were analyzed to study the optimum surface coverage for maximum sensitivity using anti-SARS-CoV-2 antibodies. Four different concentrations (i.e., 10 μg/ml, 20 μg/ml, 50 μg/ml, and 100 μg/ml) of anti-S1 (RBD) antibodies were grafted on the cantilever surface using EDC-NHS chemistry. Deflection measurements using spike proteins concentration of 1 ng/ml showed that the cantilever with antibody concentration of 20 μg/ml, 50 μg/ml and 100 μg/ml demonstrated almost the same degree of deflection (FIG. 8; Table 4). Hence, we finalized an antibody concentration of 20 μg/ml for all our deflection measurements.

TABLE 4
Different deflection values for the microcantilever sensor
with respect to different antibody concentrations

| | |
|---|---|
| Antibody concentration | Deflection |

| | | |
|---|---|---|
| 10 | μg/ml | 16.56 nm |
| 20 | μg/ml | 23.85 nm |
| 50 | μg/ml | 26.67 nm |
| 100 | μg/ml | 24.12 nm |

### Microcantilever Dimensions

The tipless gold-coated microcantilevers used for this study was 500 μm in length, 80 μm in width and 1.2 μm thick. Because a cantilever deflection also depends on geometry, all the cantilevers used in these measurements were exactly similar in their geometry.

### Complete Process Flow

FIG. 10 represents schematic of the entire process of bio-functionalization and detection.

### Patient Sample Collection and Processing Prior to Detection

Nasopharyngeal swab specimens were collected from individuals presenting to Northwestern Memorial Hospital. Per standard protocol, the nasopharyngeal swabs were placed in 1.5 to 3 mL of viral transport medium (VTM) and transported to the pathology laboratory. Prior to testing, the samples underwent a viral heat inactivation procedure by incubating them in a water bath at 65° C. for 30 minutes. Next, the samples underwent testing for the presence of the SARS-CoV-2 RNA genome by qRT-PCR (Cepheid). Positive samples were defined by their cycle threshold number (Ct) less than 45. After clinical testing was completed, positive and negative samples were stored at 4° C. for 7 days before the diagnostic testing. Patient information was removed from the sample container prior to use for research purpose. The positive samples used for the study had a spectrum of Ct values consistent with high (low Ct value) and low (high Ct value) viral load. All patient samples were typically processed prior optical detection method for microcantilever deflection monitoring. The samples were first centrifuged at 5000 rpm for 1 minute to remove mucous and other interferants. The samples were then mixed with the extraction buffer (lysis buffer) for 10 minutes and then used for the measurements. In parallel, gold-coated cantilevers were functionalized through EDC-NHS process and anchored with 50 μg/ml anti-N protein antibody.

### REFERENCES

1. WHO. Coronavirus Disease (COVID-19) Technical Guidance: Laboratory Testing for 2019-nCOV in Humans. https://www.who.int/emergencies/diseases/novel-coronavirus-2019/technical guidance/laboratory-guidance (accessed May 21, 2021).
2. Tahamtan, A., & Ardebili, A. Real-time RT-PCR in COVID-19 detection: issues affecting the results. Expert Rev. Mol. Diagn. 5, 453-454 (2020).
3. Scohy et al. Low performance of rapid antigen detection test as frontline testing for COVID-19 diagnosis. J. Clin. Virol. 129, 104455 (2020).
4. Seo, G. et al. Rapid detection of COVID-19 causative virus (SARS-CoV-2) in human nasopharyngeal swab specimens using field-effect transistor-based biosensor. ACS Nano. 14, 5135-5142 (2020).
5. Zhang, X. et al. Electrical probing of COVID-19 spike protein receptor binding domain via a graphene field-effect transistor. Preprint at https://arxiv.org/abs/2003.12529 (2020).
6. Ihling, C. et al. Mass spectrometric identification of SARS-CoV-2 proteins from gargle solution samples of COVID-19 patients. J. Proteome Res. 19, 4389-4392 (2020).
7. Kasetsirikul, S. et al. Detection of the SARS-CoV-2 humanized antibody with paper-based ELISA. Analyst. 145, 7680 (2020).
8. Zhang, L., Jackson, C. B., Mou, H. et al. SARS-CoV-2 spike-protein D614G mutation increases virion spike density and infectivity. Nat Commun. 11, 6013 (2020).
9. Hahn, G., Wu, C. M. Mutations in SARS-CoV-2 spike protein and RNA polymerasecomplex are associated with COVID-19 mortality risk. Preprint at https://doi.org/10.1101/2020.11.17.386714 (2020).
10. Agarwal, D. A. et al. Detection of heart-type fatty acid-binding protein (h-FABP) using piezoresistive polymer microcantilevers functionalized by a dry method. Appl. Nano. 8, 1031-1042 (2018).
11. Larvik, N. V., Sepenaik, M. J. & Datskos, P. G. Cantilever transducers as a platform for chemical and biological sensors. Rev. Sci. Instrum. 75, 2229 (2004).
12. Boisen, A. et al. Cantilever like micromechanical sensors. Rep. Prog. Phys. 74, 036101 (2011).
13. Agarwal, D. K, Kushagra, A., Ashwin, M., Shukla, A. S, Palaparthy, V., Sensitive detection of cardiac troponin-I protein using fabricated piezoresistive microcantilevers by a novel method of asymmetric biofunctionalization. nanotechnology 31, 115503 (2020).
14. Fritz, J. et al. Translating biomolecular recognition into nanomechanics. Science 5464, 316-318 (2000).
15. Tamayo, J. Biosensors based on nanomechanical systems. Chem. Soc. Rev. 42, 1287-1311 (2013).
16. Singh, M., Kaur, N., Comini, M. The role of self-assembled monolayers in electronic devices. J. Mater. Chem. 8, 3938-3955 (2020).
17. Viola, K. L et al. Towards non-invasive diagnostic imaging of early-stage Alzheimer's disease. Nat. Nanotechnol. 10, 91-98 (2015)
18. Samantha, D., Sarkar, A. Immobilization of bio-macromolecules on self-assembled monolayers: methods and sensor applications. Chem. Soc. Rev. 40, 2567-2592 (2011).
19. Fischer, Marcel JE. “Amine coupling through EDC/NHS: a practical approach.” In Surface plasmon resonance, 55-73. Humana Press (2010).
20. Stoney, G. G., The tension of metallic films deposited by electrolysis. Proc. Roy. Asoc. Lond. A, 82, 172-175 (1909).
21. Grant, O. C., Montgomery, D., I to, K. & Woods, R. J. Analysis of the SARS-CoV-2 spike protein glycan shield reveals implications for immune recognition. Sci. Rep. 10, 14991 (2020).
22. Wu, S. et al. Mechanism and enhancement of the surface stress caused by a small-molecule antigen and antibody binding. Biosens. Bioelctron. 48, 67-74 (2013).
23. Wu, G. et al. Origin of nanomechanical cantilever motion generated from biomolecular interactions. Proc. Natl. Acad. Sci. 4, 1560-1564 (2001).
24. Lu, R. Genomic characterisation and epidemiology of 2019 novel coronavirus: implications for virus origins and receptor binding. Lancet. 10222, 565-574 (2020).
25. E. Surkova, V. Nikolayevskyy, F. Drobniewski, False-positive COVID-19 results: hidden problems and costs. Lancet. 8, 1167-1168 (2020).
26. K. Ramdas, A. Darzi, S. Jain, Test, re-test, re-test': using inaccurate tests to greatly increase the accuracy of COVID-19 testing. Nat. Med. 26, 810-811 (2020).
27. F. B. Jhonson, Transport of viral specimens. Clin. Microbiol. Rev. 3, 120-131 (1990).
28. C. Batejat, Q. Grassin, J. C. Manuguerra, I. Leclercq, Heat inactivation of the severe acute respiratory syndrome coronavirus 2. J. Biosaf. Biosec. 3, 1-3 (2021).
29. J. BURTON ET AL., THE EFFECT OF HEAT-TREATMENT ON SARS-CoV-2 VIABILITY AND DETECTION. J. VIROL. METHODS. 290, 114087 (2021).
30. X. XIE ET AL., NEUTRALIZATION OF SARS-CoV-2 SPIKE 69/70 DELETION, E484K AND N501Y VARIANTS BY BNT162B2 VACCINE-ELICITED SERA. NAT. MED. (2021).
31. J. W. TANG, P. A. TAMBYAH, D. S. HUI, EMERGENCE OF A NEW SARS-CoV-2 VARIANT IN THE UK. J. INFECT. 82, E27-E28 (2021).
32. Q. LI ET AL., THE IMPACT OF MUTATIONS IN SARS-CoV-2 SPIKE ON VIRAL INFECTIVITY AND ANTIGENICITY. CELL 182, 1284-1294 (2020).
33. C. B. JACKSON, L. ZHANG, H. CHOE, FUNCTIONAL IMPORTANCE OF THE D614G MUTATION IN THE SARS-CoV-2 SPIKE PROTEIN. BIOCHEM. BIOPHYS. RES. COMMUN., 538, 108-115 (2021).
34. P. WENG ET AL., ANTIBODY RESISTANCE OF SARS-CoV-2 VARIANTS B.1.351 AND B.1.1.7. NATURE (2021).
35. C. R SPEAR ET AL., THE EFFECT OF SPIKE MUTATIONS ON SARS-CoV-2 NEUTRALIZATION. CELL REP. 34, 108890 (2021).
36. C. E. Gomez, B. Perdiguero, M. Esteben, Emerging SARS-CoV-2 Variants and Impact in Global Vaccination Programs against SARS-CoV-2/COVID-19. Vaccine 9, 243 (2021).
37. Larvik, N. V., Sepenaik, M. J. & Datskos, P. G. Cantilever transducers as a platform for chemical and biological sensors. Rev. Sci. Instrum. 75, 2229 (2004).
38. Fritz, J. et al. Translating biomolecular recognition into nanomechanics. Science 5464, 316-318 (2000).
39. Tamayo, J. Biosensors based on nanomechanical systems. Chem. Soc. Rev. 42, 1287-1311 (2013).

### Example 2

### Materials and Methods

Materials: SARS-CoV-2 Chimeric monoclonal antibody for Spike (S1) protein (40150-D003), SARS-CoV-2 Chimeric monoclonal antibody for Nucleocapsid (N) protein (40588-R0004), SARS-CoV-2 Spike (RBD) protein (40150-V08B2), and nucleocapsid (N) protein (40588-V08B) were all purchased from Sino Biological Inc. Phosphate Buffer Saline (PBS), PBS-Tween 20 sachets, Bovine Serum Albumin (BSA), and 11-Mercaptoundecanoic acid (MUA) were purchased from Millipore-Sigma. 1-Ethyl-3-(3-dimethylaminopropyl) carbodiimide and Sulfo-NHS were received from Thermo Fisher Scientific. For Influenza A virus detection, Mouse monoclonal antibody to Influenza A virus Nucleoprotein (ab128193) was used. Rabbit polyclonal antibody to H1N1 virus Nucleocapsid (ab104870) was used for swine flu (H1N1) detection. Rabbit polyclonal to Mycoplasma pneumonia antibody (ab53600) was used for the detection of Pneumonia. Mouse polyclonal to Enterovirus 71 (ab169442) was used for Enterovirus detection. All the antibodies for the detection of Influenza A, H1N1, Enterovirus and Pneumonia were procured from Abcam. Delta variants detection was carried out using in-house developed highly specific rabbit monoclonal antibodies against delta variants.

Functionalization of the MOSFET chips: MOSFET-embedded microcantilever chips were prepared for antibody functionalization by first blocking (passivating) the SiN sides of the cantilevers to reduce the incidence of non-specific binding during antibody and antigen exposure. Chip surfaces were cleaned by washing sequentially with acetone, isopropanol, and methanol. Chips were than briefly treated with piranha (10 seconds) followed by sequential washing with de-ionized water and ethanol.

Electronic measurements of the transistor characteristics were carried out using a Keithley 4200 semiconductor characterization system. The observed changes in the drain current at fixed gate bias and sweeping the drain voltage demonstrate the modulation of channel current with surface stress (generated due to biomolecular interactions) due to microcantilever bending. The change in drain current of the transistor results from the modulation of channel mobility because of surface stress, which increases the channel resistance. The mobility change may also arise from the changes in the interface charge densities, generation of trap-states, band structure alteration and generation of shallow defects due to localized bending stress. MOSFET transistors were passivated with silicon nitride thin coating and electrical contacts were isolated for the binding experiments in the fluidic environment.

The MOSFET microcantilever devices were first immersed in 10 mM ethanolic solution of 11-Mercaptoundecanoic acid (MUA) to attach Carboxylic moieties on the cantilever surface and then rinsed through Ethanol solution. The grafted —COOH groups were activated by putting the devices in solution mixture of 5 mM Carbodiimide EDC and 5 mM Sulfo-NHS in DI water for an hour followed by rinsing with DI water. The devices were then covalently immobilized by SARS-CoV-2 antibodies (20 μg/ml) against nucleocapsid protein by incubating overnight in an antibody solution prepared in PBS and 0.05% BSA (pH=7.4). Functionalized chips were washed repeatedly with PBS-Tween 20 solution and stored in PBS until further analysis. The detailed chemical functionalization process of MOSFET devices has been shown in FIG. 2.

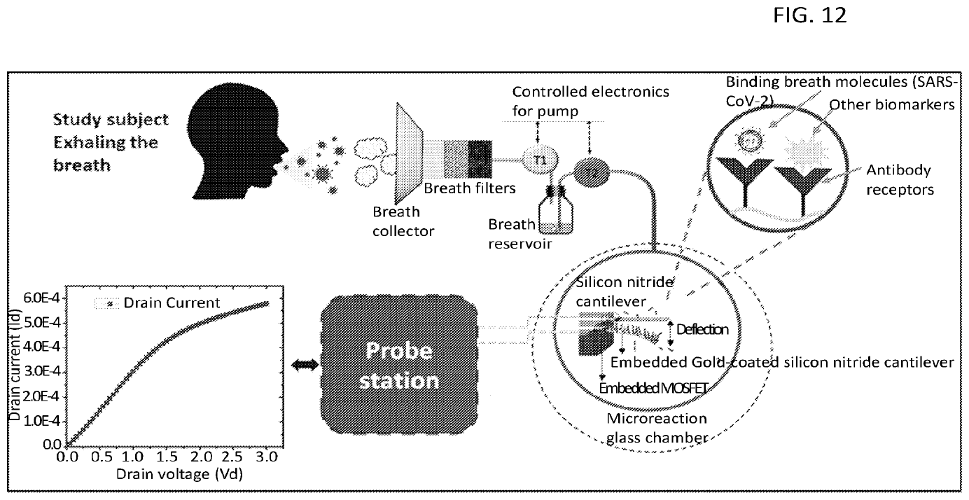

Breath sampling and measurements: Breath detection was carried out by using an in-house built breath collection chamber/analyzer (FIG. 12). The complete set-up includes a breath collector, breath filters, pump with the controlled electronics, and a micro reaction glass chamber with the externally connected parametric analyzer. The breath samples were collected from the patient's (COVID positive) and healthy individuals (negative) who were hospitalized during the delta variant pandemic wave. All samples were collected at Post graduate Institute of Medical Sciences in India. Samples collected from patients were stored in designated storage facility within the hospital. The filtered patients' exhaled breath was first collected into a breath reservoir and then filled into the sterile Tedlar® bags since they are easier to transport and cost effective^{42,43} New and separate set of collection bags were used for every patient's collected breath samples. The bag was tightly connected to the expiratory limb of the ventilator. Prior to sampling, all the Tedlar® bags were flushed with nitrogen and vacuum evacuated several times to reduce background interference. The average sampling time for exhaled breath was fixed to 25-30 seconds. Once the bags were filled, valves were closed and removed from the ventilator. The overall composition of the patients samples we collected obviously have different gaseous components (including volatile organic compounds=VOCs). However, the primary objective here was to detect viral particle load and associated antigens though specific antibody-binding. All the measurements were performed in the breath-based VOCs detection facility at the hospital which has all the standard measurements protocols in place for breath-based detection of viral moieties. All the breath samples collected from patients were undergone RT-PCR test to identify the level of infection.

### Patient Sample Collection and Processing (Swab-Based Detection)

Nasopharyngeal swab specimens were freshly collected from individuals presenting to Northwestern Memorial Hospital and transported to the pathology laboratory. Prior to testing, the samples underwent a viral heat inactivation procedure by incubating them in a water bath at 65° C. for 30 minutes. Next, the samples underwent testing for the presence of the SARS-CoV-2 RNA genome by qRT-PCR (Cepheid). Positive samples were defined by their cycle threshold number (Ct) less than 45. After clinical testing was completed, positive and negative samples were stored at 4° C. for 7 days before the diagnostic testing. Patient information was removed from the sample container prior to use for research purpose. The positive samples used for the study had a spectrum of Ct values consistent with high (low Ct value) and low (high Ct value) viral load. All patient samples were typically processed prior optical detection method for microcantilever deflection monitoring. The samples were first centrifuged at 5000 rpm for 1 minute to remove mucous and other interferants. The samples were then mixed with the extraction buffer (lysis buffer) for 10 minutes and then used for the measurements.

### Results and Discussion

Prior to breath based detection, we optimized microcantilever measurements using patients' nasopharyngeal swab samples pre-analyzed by qRT-PCR. These measurements helped establish MOSFET drain current sensitivity, optimized affinity coating coverage and associated parameters. We then confirmed the notable consistency between MOSFET drain current change (due to microcantilever deflection) and the Ct values (i.e., the number of PCR cycles that are needed for confirmation of viral genome presence). This combination of high sensitivity coupled to very high sensitivity to viral load prompted us to implement and subsequently validate breath sampling for Covid diagnosis.

### SARS-CoV-2 Detection Through Nasopharyngeal Samples: Optimization of Drain Current for the Nanomechanical Signal Transduction

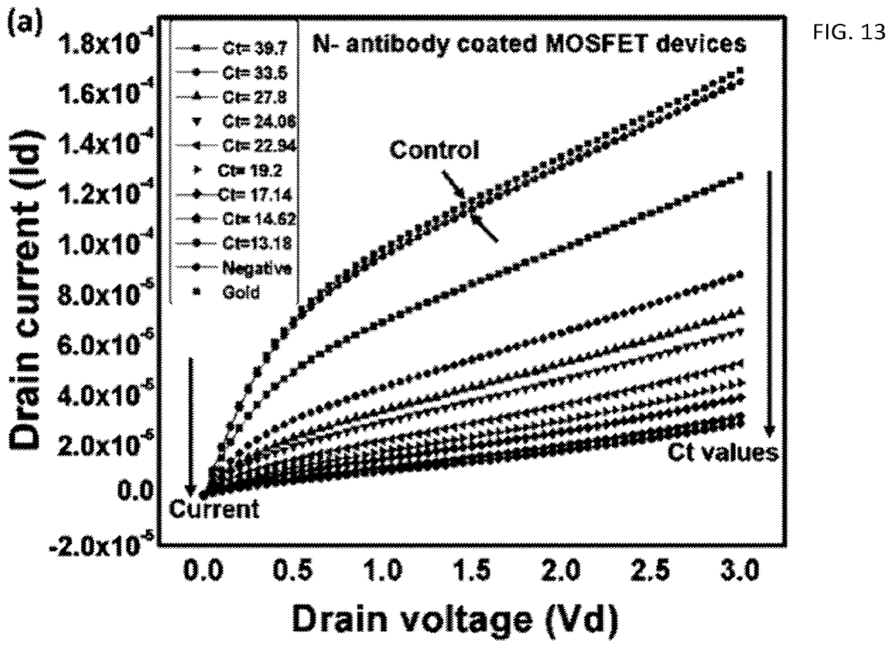

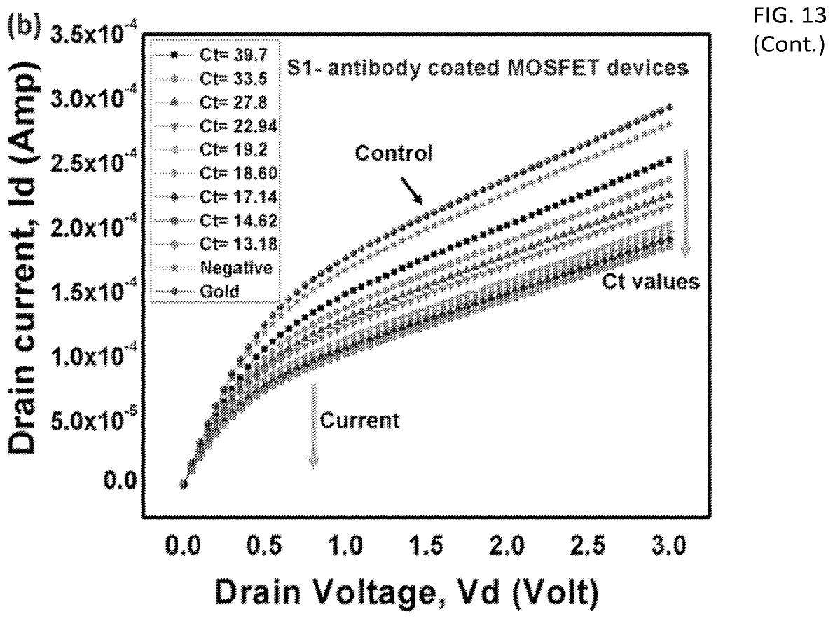

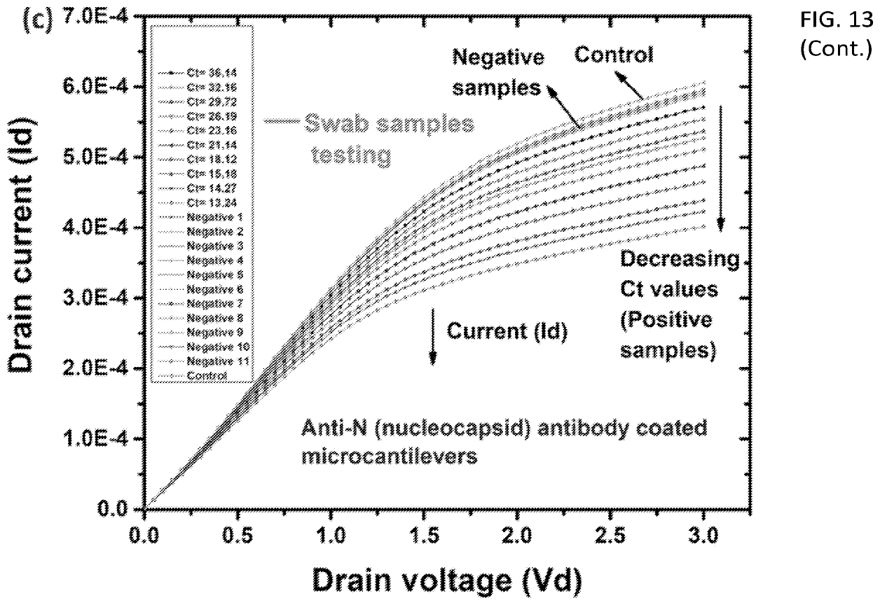

We first conducted optimized MOSFET detection of SARS-CoV-2 through patients' nasopharyngeal swab specimens collected freshly from individuals admitted to Northwestern Memorial Hospital, Chicago. Prior to testing, typical sampling and handling procedures consistent with other test methods were employed for both COVID positive and healthy individuals (negative samples). For example, it followed a viral heat inactivation procedure for patients' samples by incubating them in a water bath at 65° C. for 30 minutes. The positive samples used for the study had a spectrum of Ct values consistent with high viral load (low Ct value to achieve detection) and low viral load (high Ct value). The drain current measurements were carried out with respect to the differing Ct values from different COVID positive patient samples (n=19) along with negative patient samples. As mentioned earlier, the nasopharyngeal swab samples were collected from the patients and Ct values were pre-determined with qRT-PCR. In the first set of measurements (FIGS. 13a and 13b), nine patients' samples with a Ct value varying from 13.18 to 39.7 were employed, the latter values are considered to be clinical limit of detection of PCR, indicating extremely low viral load (approximately 100 viral particles) or minimal antigen presence.

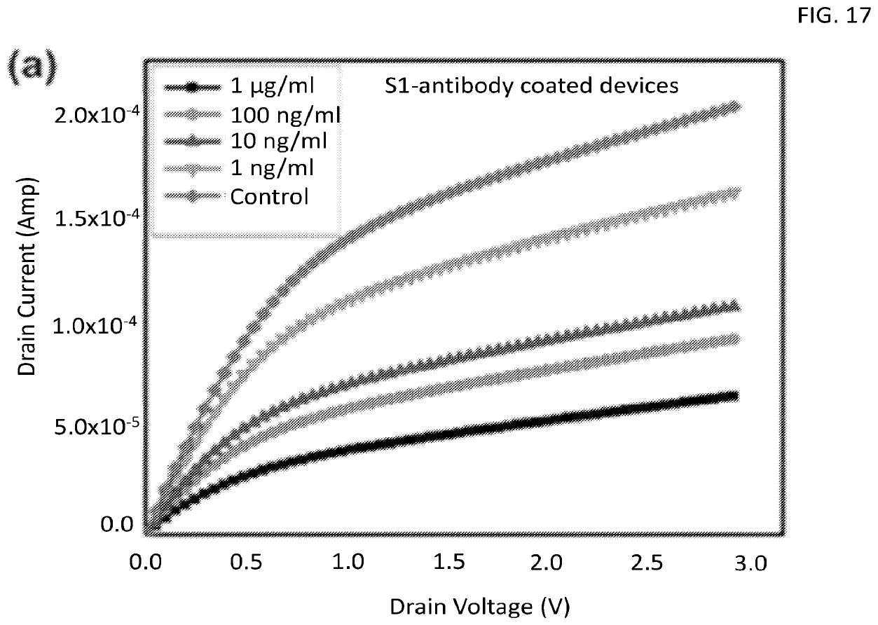

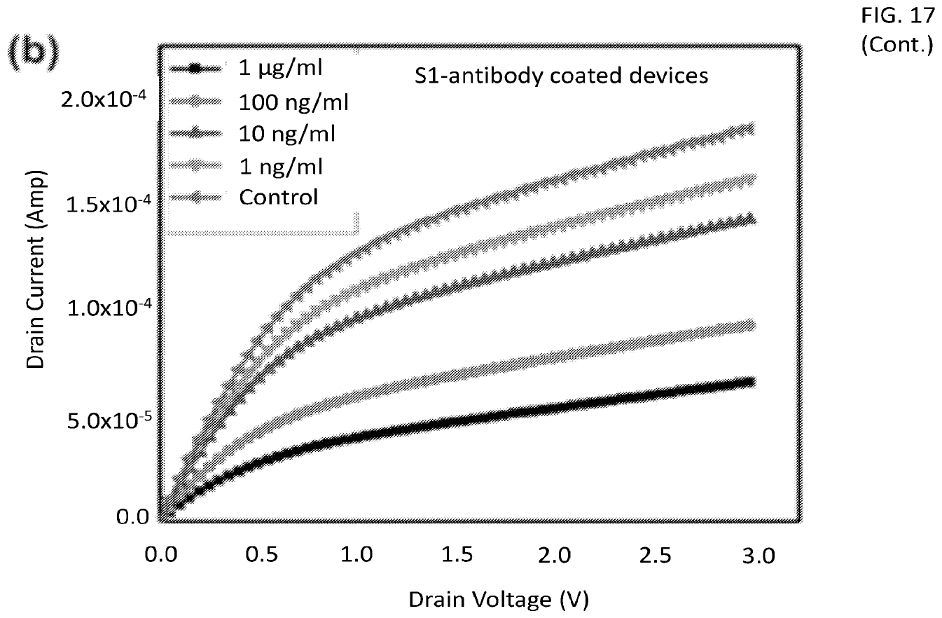

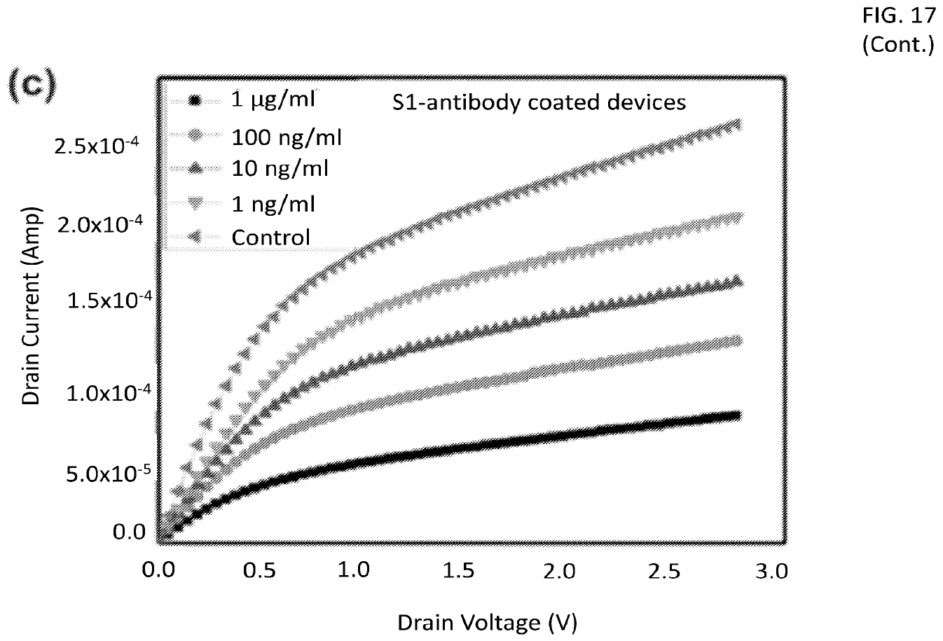

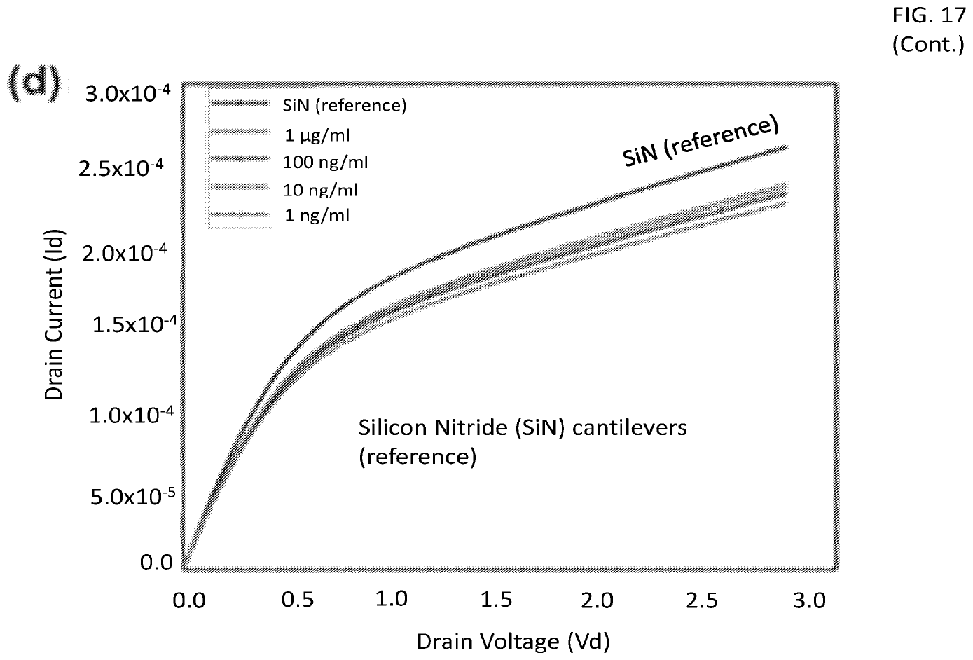

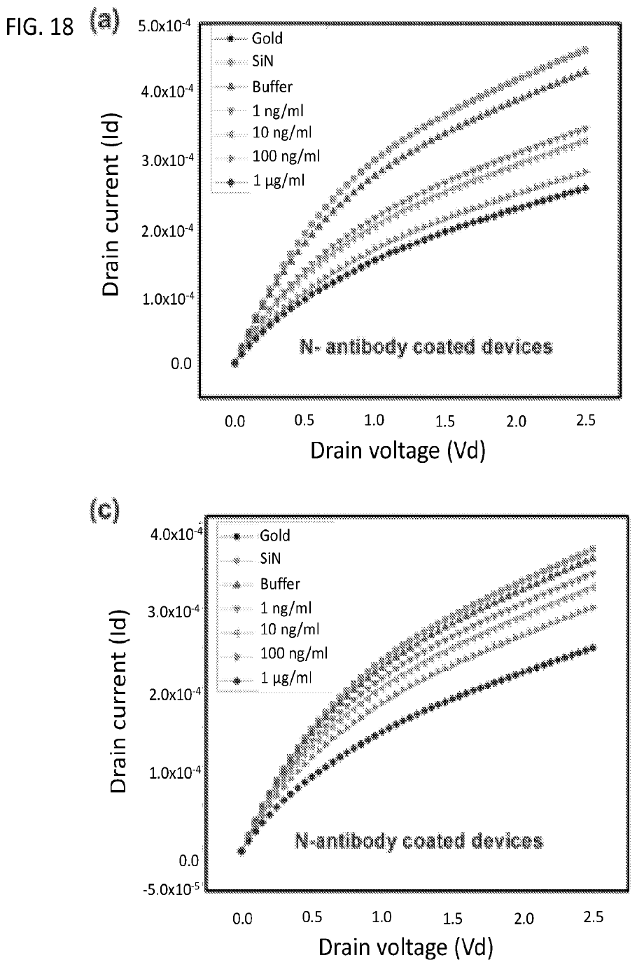

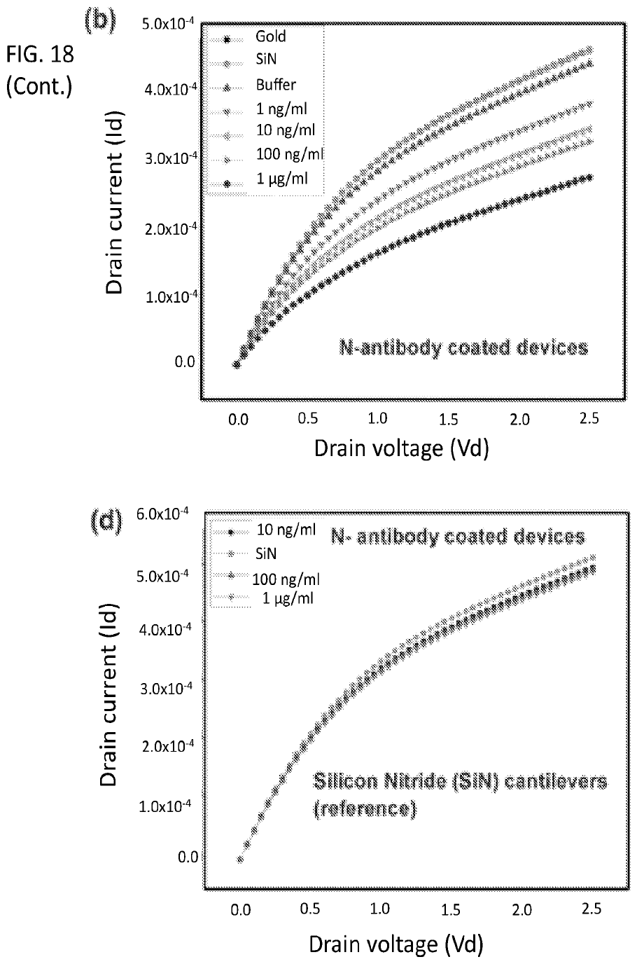

The MOSFET drain current measurements were performed by using two different sets of MOSFET devices coated with antibodies for SARS-CoV-2 N and S1 proteins to compare the current change. As seen from the FIG. 13, separation of different curves for different Ct values were significantly large in anti-N-antibody coated devices compared to anti-S1-antibody coated MOSFET devices, resulting less change in the current in latter's case. The extent of bending has many complex considerations. Perhaps part of it may be rationalized by the fact that SARS-CoV-2 sheds more nucleocapsid antigen proteins than compared to spike proteins. There was no significant change in the current for the control sample in both cases within error bars. Here, the data collected from the gold coated cantilevers with conjugated antibodies (with no antibody-antigen interaction) is used as the control sample. Prior to patient-sample based study, we had earlier demonstrated SARS-CoV-2 S1 and N protein detection on biofunctionalized MOSFET devices with the corresponding antibodies (FIGS. 17 and 18).

Following the SARS-CoV-2 detection using patients' swab samples, another study was conducted on the patients who further volunteered for breath detection. Here samples were collected from 10 positive and 10 negative patients with different Ct values and current measurement was conducted. As shown in FIG. 13 (c), the highest Ct value (36.14) showed the maximum current whereas the lowest Ct value (13.24) exhibited the minimal current change. All the negative patients' samples showed no significant change and in fact these curves can be seen on top of each other within error bar in the figure. Both the studies conducted on two different sets of samples showed the effectiveness of MOSFET sensor in rapid detection of SARS-CoV-2 in nasopharyngeal swab samples.

After successfully demonstrating MOSFET electronic detection of viral antigens with great sensitivity and requisite specificity, we explored whether the sensitivity could be adequate as a step towards breath-based detection. The MOSFET possesses sufficient signal to noise ratio (S/N ratio) that it may be possible to detect even lower concentration of viral antigens through exhaled breath as we show next.

“Breathalyzer” Test for COVID-19: Non-Invasive Detection of SARS-CoV-2 Antigens with Breath Sampling

The nasopharyngeal swab results demonstrate fast, sensitive, and specific antigen detection by the MOSFET-embedded microcantilever assay. Given the sensitivity of our microcantilever detection scheme down to just 100+ viral particles, it opens prospects for detecting via breath sampling. The analysis of antigen-based analytes in exhaled breath samples would represent potentially an entirely new paradigm in diagnostics because it is a noninvasive, may be sensitive to early-stage diagnosis and potentially inexpensive way to detect respiratory threat exposure. The major benefits of breath analysis with respect to its clinical application are its non-invasive nature, acceptable for most vulnerable patients, ease of use and rapid. Analysis from breath requires very short time and avoid any complex sample preparation procedures used in other methods.

We implemented our MOSFET-embedded cantilever-based diagnostics system for detecting COVID-19 via breath since MOSFET with swab samples demonstrated a great sensitivity which may be amenable to breath sampling as well. This integrated technology promises to provide a turn-key non-invasive diagnostics system for rapid COVID-19 detection via breath. These MOSFET chips were used to distinguish different level of pathogenicity (viral load) among the patients volunteered for the breath-based SARS-CoV-2 detection study.

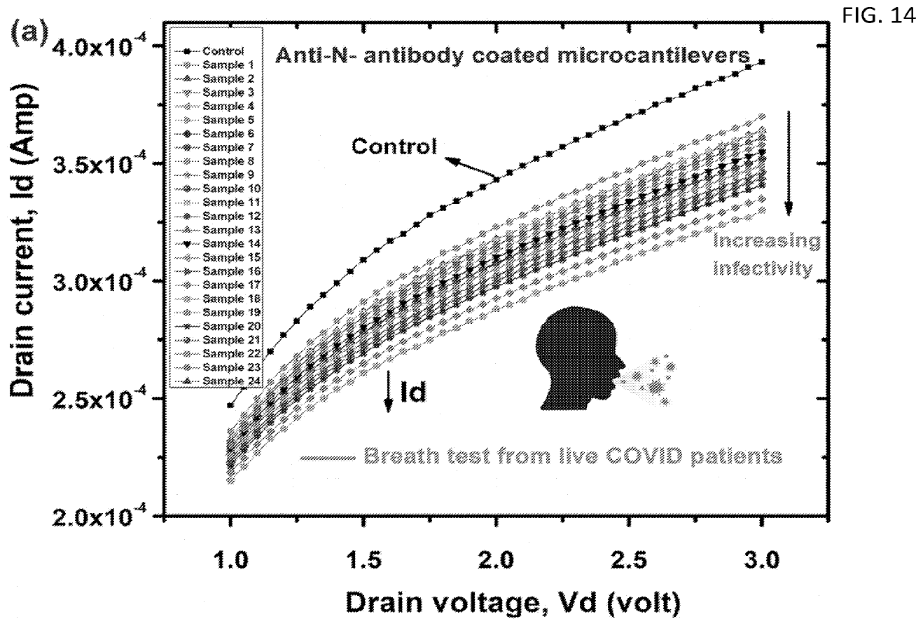

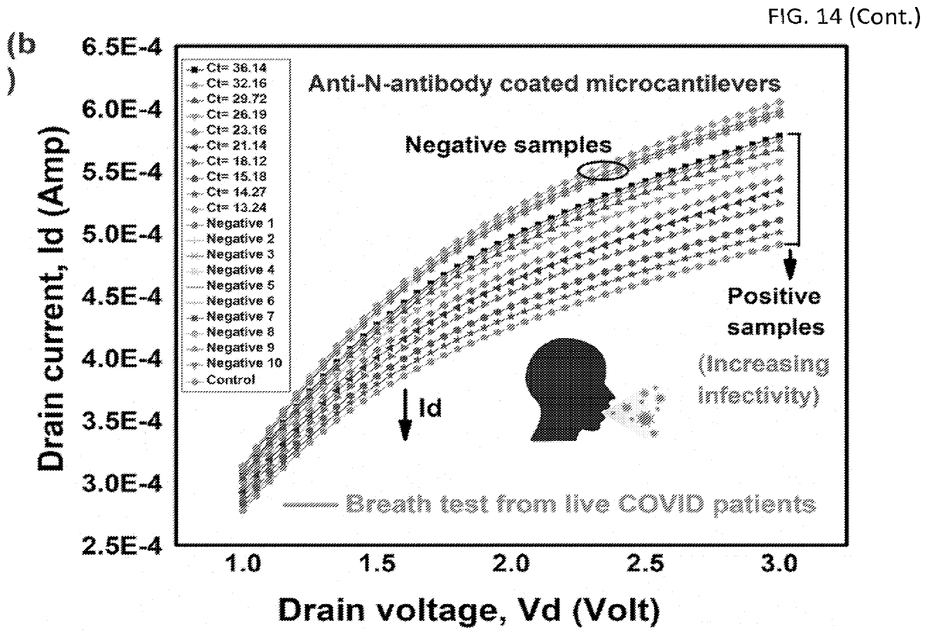

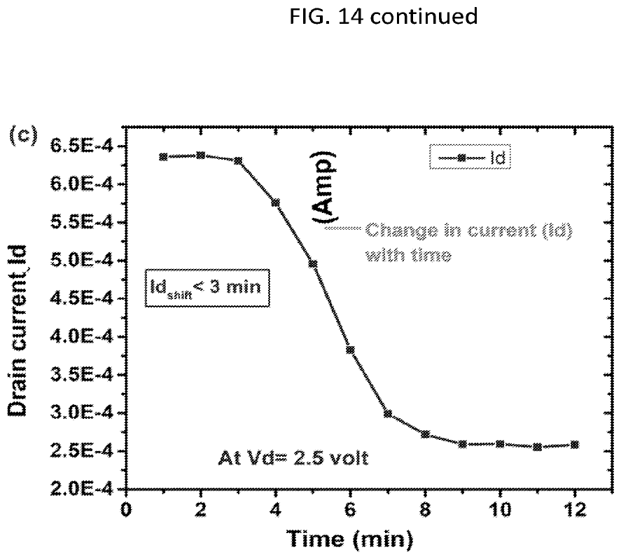

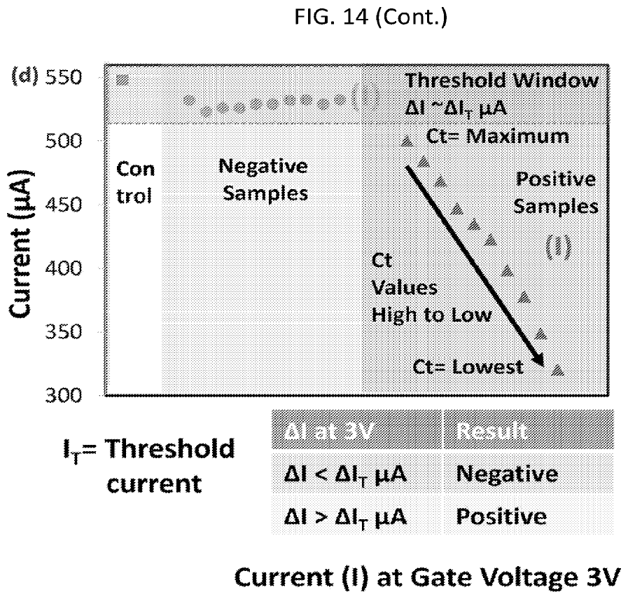

FIG. 14a shows direct detection of SARS-CoV-2 from infected patients' breath samples. As the infection in the patient increases in the sense of higher viral load (thus the antigens), the change in the drain current is relatively large compared to control sample. The control sample here represents to a silicon nitride (without gold coating) cantilever. The gate voltage was kept at 2V throughout all the experiments. The drain current decreases as the infectivity (viral load) increases due to large change in the compressive surface stress of the cantilever which results in significant bending. No change in the drain current, within errors and noise, was observed for SiNx/reference cantilevers.

Similar experiments were performed for detection of SARS-CoV-2 using patients' breath (as shown in FIG. 14b) with a combination of both positive and negative samples (predetermined with qRT-PCR for different level of viral load/Ct values). The COVID positive patients volunteered here, as presented in FIG. 14b, earlier used for swab-based measurement (FIG. 13c). Likewise, the gate voltage was kept constant at 2V in these experiments as well.

These results strongly demonstrate high sensitivity of the non-invasive detection method, and we are able to detect to the level the RT-PCR can do. All the patients underwent standard RT-PCR detection first to determine the level of the viral infection (Ct values determination). Here, patient with the lowest Ct value (13.24) exhibited the minimum drain current (bottom curve line), whereas the exhaled breath coming from the patient with the highest Ct value (36.14) shows the maximum drain current (black curve line). FIG. 14c shows the dynamic response of the cantilever to viral load over a period of time. Our data demonstrates that the shift in the drain current occurs in less than 3 minutes of exposure of the exhaled breath, making it an ultra-rapid and sensitive detection method. FIG. 14d, represents the MOSFET sensor as a switch which shows the positive detection signal above a certain threshold of viral load (red zone) and below which it exhibits a linear regime (green zone).

### Breath-Based Multiplexed Detection of Viral Borne Diseases

Since Covid-19 and other respiratory disease symptoms often overlap and can be mistaken for one or the other, we attempted to demonstrate that breath sampling can provide sufficient discrimination across a broader swathe of pathogen detection. The MOSFET/cantilever technology is amenable to not only massively parallel and multiplexed sensing but fully compatible with microfluidics with all the advantages of the commercially versatile microelectronics platform.

In this study, array of ten silicon based MOSFET-embedded direct readout cantilevers were used to detect different respiratory disease pathogen biomarkers through exhaled breath from different patients. Here, subjects suffering from COVID-19 symptoms (Delta variant), Influenza A, HIN1 (Swine flu), Enterovirus mediated disease, and Pneumonia volunteered for this study along with some healthy patients (control). The arrays were prior bio-functionalized using different capillaries (channels) filled with individual antibody solution against these viral antigens. The antibody coated MOSFET chips were then placed inside the breath-based measurement setup and direct differential readout was carried out to minimize any false positives. Differential measurements of sensor versus reference microcantilever responses are very important to eliminate false positives as bending signal can be influenced by temperature variations as well as non-specific reactions/binding.

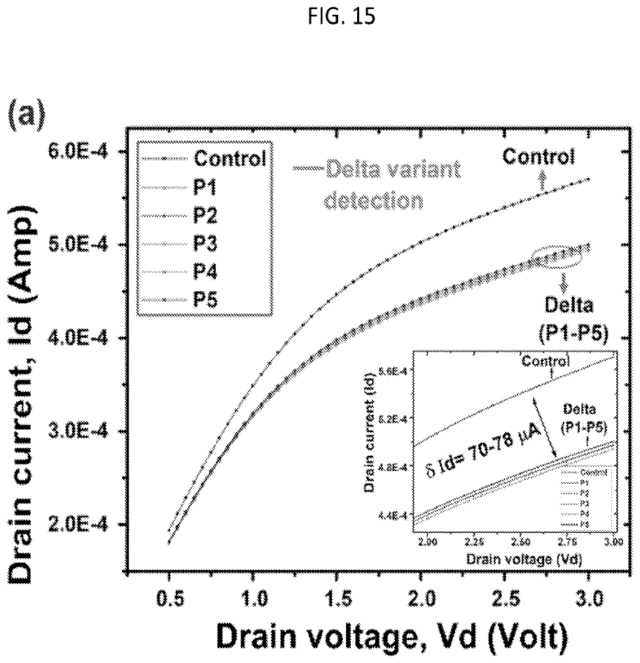

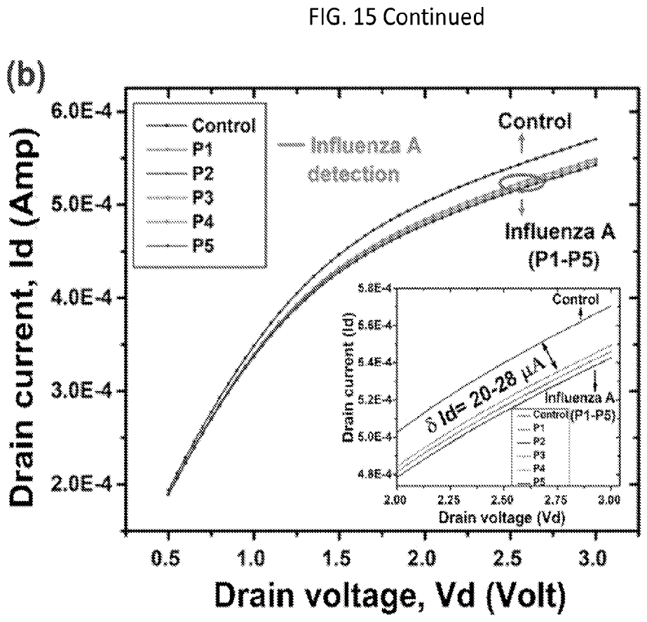

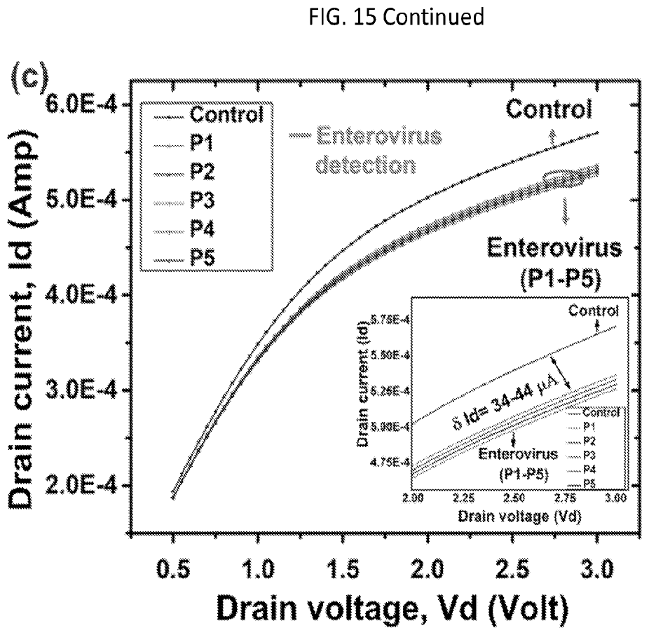

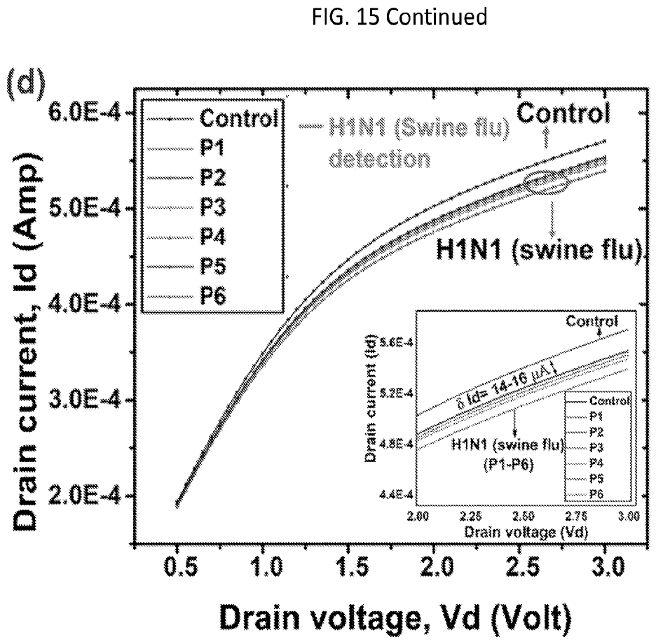

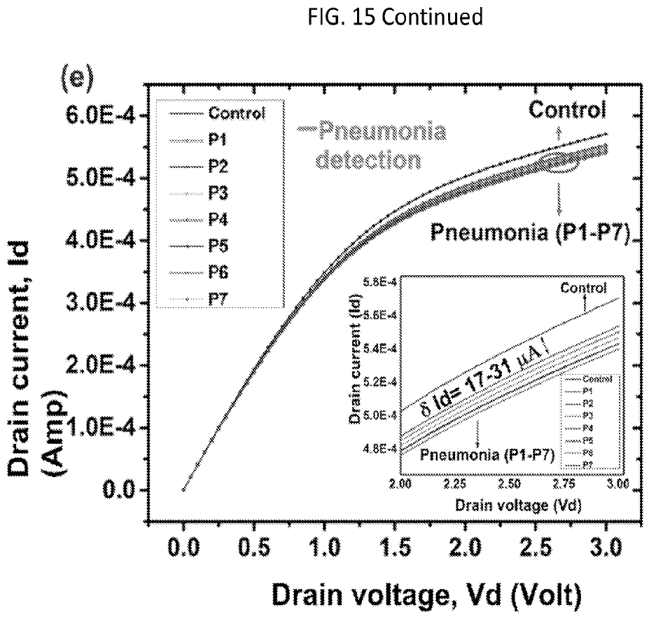

The current modulation data mediated through antibody-antigen based interaction were collected for different subjects under study. As seen from the figure that SARS-CoV-2 variant (Delta) yielded the maximum change in the drain current (70-78 μA) among different patients volunteered indicating typically higher viral load compared to other subjects under study. All the measurements were performed simultaneously. The results in FIG. 15 provides tantalizing prospects for multiplexed detection capabilities of this technology via breath.

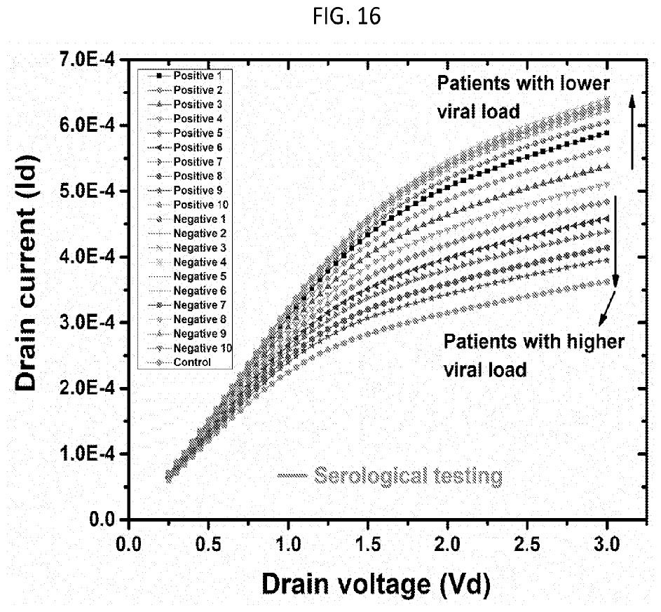

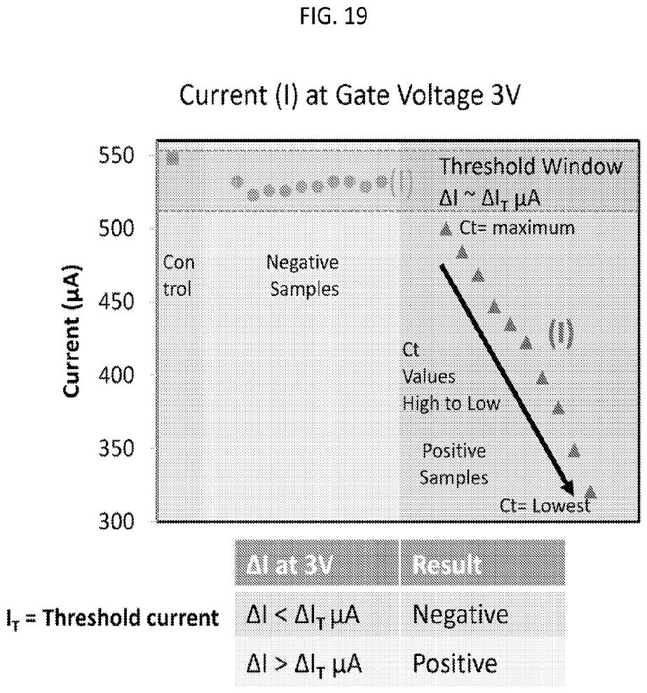

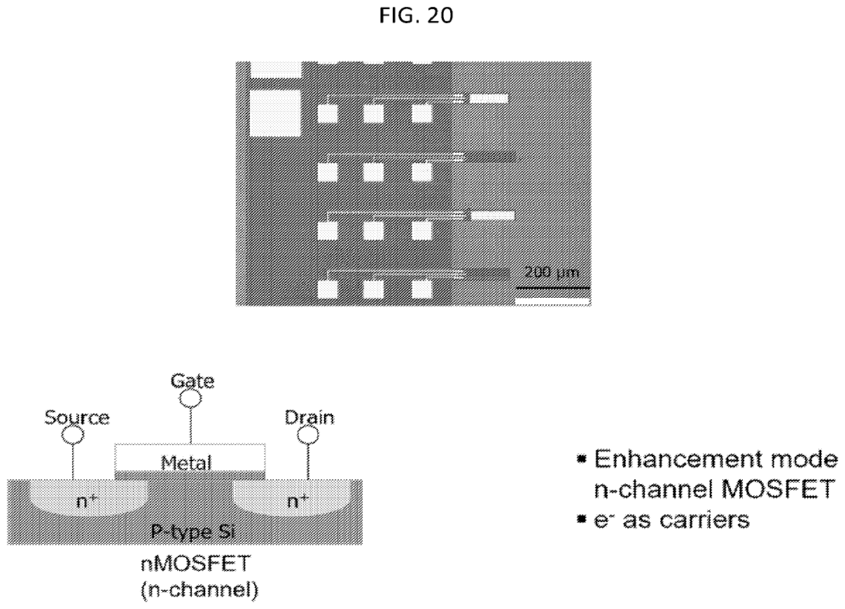

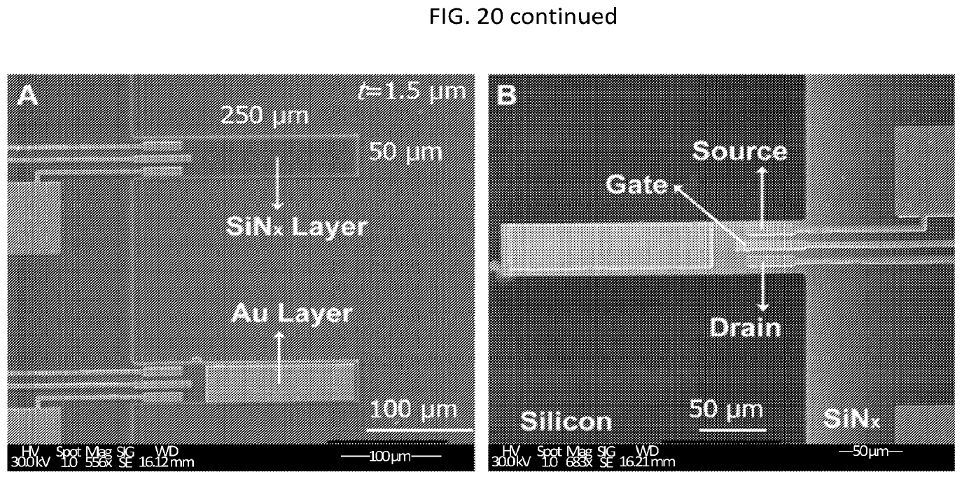

Table 5. Shows the details about the COVID patients volunteered for swab and breath-based study. In total, 68 COVID positive patients and 32 COVID negative patients were tested for breath, nasopharyngeal swab, and serum samples (serological testing,). The serological testing data have been presented in FIG. 16. It also summarizes the sensitivity, accuracy, and detection time of our developed sensor technology. The above data shows that the MOSFET platform can be developed into a point-of-care hand-held device which can act as a switch for COVID monitoring working above a certain threshold (See FIG. 19).

TABLE 5
Details of COVID patients volunteered for this study.

| | COVID | COVID | | | | |
|---|---|---|---|---|---|---|
| | positive | negative | | | Detection | |
| Sample types | patients (n) | patients (n) | Sensitivity | Accuracy | time | |
| Breath | 39 | 10 | ≥99% | ≈100 | ≤3 | min |
| Nasopharyngeal | 19 | 12 | ≥98-100% | ≈98-100 | ≈3-5 | min |
| swab | | | | | | |
| Serum (S1) | 10 | 10 | ≥96-100% | ≈98 | ≤5 | min |

The results discussed here indicate the detection capabilities of the integrated MOSFET device as a potential platform for a non-invasive, ultra-rapid (breath to signal≤3 min), and highly selective breath-based antigen test for SARS-CoV-2 and other related respiratory pathogens. These results provide impetus for building towards miniature and turnkey breath sampling “portable” system that is integrated with a mobile phone device.

### Summary & Conclusions

We demonstrate MOSFET based electrical detection of SARS-CoV-2 in patient through ‘breath’ as a sampling method with high degree of sensitivity, specificity, and selectivity in just a few minutes (“breath to signal” in less than 3 mins). In addition, we also report multiplexed detection capabilities by detecting series of respiratory virus simultaneously along with SARS-CoV-2 Delta variant. We have conducted additional correlative study with swab and serological patient's samples to demonstrate the high sensitivity of our diagnostics system and compatibility with other sample collection methods.

We believe, the non-invasive rapid detection method through exhaled breath will not only reduce unnecessary viral exposure but also availability to vulnerable and underserved population and children's going back to school. The ability to detect the infection before the symptoms appears because of its high sensitivity will significantly reduce the spread of this contagious disease especially when the active variants are moving around.

### REFERENCES

1. Yu, L. et al. Rapid detection of COVID-19 coronavirus using a reverse transcriptional loop-mediated isothermal amplification (RT-LAMP) diagnostic platform. Clin. Chem. 66, 975-977 (2020).
2. Kevadiya, B. D. et al. Diagnostics for SARS-CoV-2 infections. Nat. Mater. 20, 593-605 (2021).
3. Lamb, L. E., Bartolone, S. N., Ward, E. and Chancellor, M. B. Rapid detection of novel coronavirus/Severe Acute Respiratory Syndrome Coronavirus 2 (SARS-CoV-2) by reverse transcription-loop-mediated isothermal amplification. Lancet 15, e023468 (2020).
4. Wang, D. et al. Development of a real-time loop-mediated isothermal amplification (LAMP) assay and visual LAMP assay for detection of African swine fever virus (ASFV). J. Virol. Methods. 276, 113775 (2020).
5. Amer, H. M. et al. A new approach for diagnosis of bovine coronavirus using a reverse transcription recombinase polymerase amplification assay. J. Virol. Methods. 193, 337-340 (2013).
6. Broughton, J. P. et al. Rapid detection of 2019 novel coronavirus SARS-CoV-2 using a CRISPR-based DETECTR lateral flow assay. medRxiv. 2020.
7. Bacura, A. S. et al. Current status of the lateral flow immunoassay for the detection of SARS-CoV-2 in nasopharyngeal swabs. Biochem. Med. 31, 020601(2021).
8. Andryukov, B. G. Six decades of lateral flow immunoassay: from determining metabolic markers to diagnosing COVID-19. AIMS Microbiol. 6, 280-304, 2020.
9. Grant, B. D. et al. SARS-CoV-2 Coronavirus nucleocapsid antigen-detecting half-strip lateral flow assay toward the development of point of care tests using commercially available reagents. Anal. Chem. 92, 11305-11309 (2020).
10. Chu, D. K. W. et al. Molecular diagnosis of a novel coronavirus (2019-nCOV) causing an outbreak of pneumonia. Clin. Chem. 66, 549-555 (2020).
11. Capobianchi, M. R. et al. Molecular characterization of SARS-CoV-2 from the first case of COVID-19 in Italy. Clin. Microbiol. Infect. 26, 954-956 (2020).
12. Itoh, K. et al. False positive results in severe acute respiratory coronavirus 2 (SARSCoV-2) rapid antigen tests for inpatients. J Infect Chemother, 27, 1089-1021 (2021).
13. Peeling, R. W., Olliaro, P. L., Boeras, D. I. and Fongwen, N. Scaling up COVID-19 rapid antigen tests: promises and challenges. Lancet. Infect. Dis. (2021).
14. Mak, G. CK. Et al. Evaluation of rapid antigen test for detection of SARS-CoV-2 virus. J. Clin. Virol. 129, 104500 (2020).
15. Ikeda, M. N. et al. Clinical evaluation of self-collected saliva by quantitative reverse transcription-PCR (RT-qPCR), direct RT-qPCR, reverse transcription-loop-mediated isothermal amplification, and a rapid antigen test to diagnose COVID-19. J. Clin. Microbiol. 58, e01438-20 (2020).
16. Scohy, A. et al. Low performance of rapid antigen detection test as frontline testing for COVID-19 diagnosis. J. Clin. Virol. 129, 104455 (2020).
17. Ogawa, T. et al. Another false-positive problem for a SARS-CoV-2 antigen test in Japan. J. Clin. Virol. 131, 104612 (2020).
18. Y. Y. Broza, R. Vishinkin, O. Barash, M. K. Nakhleh and H. Haick. Synergy between nanomaterials and volatile organic compounds for non-invasive medical evaluation. Royal Society of Chemistry, 2018; Vol. 47.
19. Y. Y. Broza, P. Mochalski, V. Ruzsanyi, A. Amann and H. Haick, Hybrid Volatolomics and disease detection. Angew. Chem., Int. Ed. 2015, 54 (38), 11036-11048.
20. J. Schijven, L. C. Vermeulen, A. Swart, A. Meijer, E. Duizer, A. M. de Roda Husman. Exposure assessment of airborne transmission of SARS-CoV-2 via breathing, speaking, coughing and sneezing. medRxiv 2020, DOI: 10.1101/2020.07.02.20144832.
21. D. Ruszkiewicz, D. Sanders, R. O'Brien, F. Hempel, M. J. Reed, A. C. Reiepe, K. Bailie, E. Brodrick, K. Darnley and R. Ellerkmann. Diagnosis of COVID-19 by analysis of breath with gas chromatography-Ion mobility spectrometry—a feasibility study. EclinicalMedicine 2020, 29-30, 100609.
22. J. Ma, X. Qi, H. Chen, X. Li, Z. Zhang, H. Wang, L. Sun, L. Zhang, J. Guo, L. Morawska. Coronavirus disease 2019 patients in earlier stages exhaled millions of severe acute respiratory syndrome coronavirus 2 per hour. Clin. Infect. Dis. 2020, 14-16.
23. D. Z. Guo, Y. Z. Wang, F. S. Zhang, X. Li, L. Li, C. Li, Y. Cui, R. B. Fu, Y. Z. Dong, X. Y. Chi. Aerosol and surface distribution of severe acute respiratory syndrome coronavirus 2 in hospital wards, Wuhan, China, 2020. Emerging Infect. Dis. 2020, 26 (7), 1583-1591.
24. J. L. Santarpia, D. N. Rivera, V. L. Herrera, M. J. Morwitzer, H. M. Creager, G. W. Santarpia, K. K. Crown, D. Brett-Major, E. R. Schnaubelt, M. J. Broadhurst. Aerosol and surface transmission potential of SARS-CoV-2. Sci. Rep. 2020, DOI: 10.1038/s41598-020-69286-3.
25. Y. Liu, Z. Ning, Y. Chen, Guo, Y. Liu, N. K. Gali, L. Sun, Y. Duan, J. Cai, D. Westerdahl. Aerodynamic analysis of SARS-CoV-2 in two Wuhan hospitals, Nature 2020, 582 (7813), 557-560.
26. G. Giovannini, H. Hossam, and D. Garoli. Detecting COVID-19 from breath: A Game changer for a big challenge, ACS Sens. 2021, 6, 1408-1417.
27. Malik, M., Kunze, A-C., Bahmer, T., Rosenthal, S. H., Kunze, T., SARS-CoV-2: Viral Loads of Exhaled Breath and Oronasopharyngeal Specimens in Hospitalized Patients with COVID-19, International Journal of Infectious Diseases 110 (2021) 105-110.
28. Seo, G., Lee, G., Kim, M. J., Baik, S. H., Choi, M., Ku, K. B., Rapid detection of COVID-19 causative virus (SARS-CoV-2) in human nasopharyngeal swab Specimens using Field-Effect Transistor-based biosensor, ACS Nano, 14, 5135-5142 (2020).
29. Sengupta, J., Hussain, C. M., Graphene-based field-effect transistor biosensors for the rapid detection and analysis of viruses: A perspective in view of COVID-19, Carbon Trends, 2, 100011 (2021).
30. Hafshejani, P. F et al., Two-dimensional material-based Field Effect Transistor biosensing for detecting COVID-19 virus (SARS-CoV-2), ACS Nano, 15, 7, 11461-11469 (2021).
31. Agarwal, D. A. et al. Detection of heart-type fatty acid-binding protein (h-FABP) using piezoresistive polymer microcantilevers functionalized by a dry method. Appl. Nano. 8, 1031-1042 (2018).
32. Larvik, N. V., Sepenaik, M. J. & Datskos, P. G. Cantilever transducers as a platform for chemical and biological sensors. Rev. Sci. Instrum. 75, 2229 (2004).
33. Boisen, A. et al. Cantilever like micromechanical sensors. Rep. Prog. Phys. 74, 036101 (2011).
34. Agarwal, D. K, Kushagra, A., Ashwin, M., Shukla, A. S, Palaparthy, V., Sensitive detection of cardiac troponin-I protein using fabricated piezoresistive microcantilevers by a novel method of asymmetric biofunctionalization. nanotechnology 31, 115503 (2020).
35. Fritz, J. et al. Translating biomolecular recognition into nanomechanics. Science 5464, 316-318 (2000).
36. Tamayo, J. Biosensors based on nanomechanical systems. Chem. Soc. Rev. 42, 1287-1311 (2013).
37. Agarwal, D. K. et al., Highly Sensitive and Ultra-Rapid Antigen-based Detection of SARS-CoV-2 in Nasopharyngeal Swab Specimens by Microcantilever Sensors, Biosens. Bioelectron. 113647 (2021).
38. Shekhawat, G., Tark, S H. and Dravid, V. P. MOSFET-Embedded Microcantilevers for Measuring Deflection in Biomolecular Sensors. Science. 311, 1592-1595 (2006).
39. Salwa Mustafa, F. Tulip, Ida lee, S. Kamrul Islam and G. Shekhawat, Integrated MOSFET-Embedded Cantilever based Biosensor for Detection of Anthrax Simulant. IEEE Electron Device Letters, Vo. 32 (3), 2011, pp. 448. https://10.1109/LED.2010.2098015.
40. Gajendra, S. and Vinayak, D., Nanomechanical Sensors: bent on detecting cancer, Nature Nanotechnology 8(2), 77 (2013).
41. Gajendra, S. and Vinayak D., Biosensors: Micro-cantilevers to lift molecules, Nature Nanotechnology 10, 830 (2015).
42. Franchina, F. A. et al., Towards the use of breath for detecting mycobacterial infection: a case study in murine model, J. Breath Res. 12, 026008 (2018).
43. Steeghs, M. M. L, Cristescu, S. M., Harren, F. J. M., The suitability of Tedlar bags for breath sampling in medical diagnostics research, Physiol. Meas. 28, 73 (2006)
44. Sharma. P., Chopra, A., Chaudhary, S., Suri, C. R. Bio-nanomechanical Detection of Diabetic Marker HbA1c. BioNanoSci. 2, 179-184 (2012).
45. https://www.thermofisher.com/order/catalog/product/26102 #/26102.

1. A nanomechanical sensor comprising an antibody-functionalized microcantilever.

2. The sensor of claim 1, wherein the antibody-functionalized microcantilever comprises a microcantilever, an antibody configured to bind a target antigen or an antigen configured to bind a target antibody, and a tether covalently tethering the antibody or the antigen to the microcantilever.

3. The sensor of claim 2, wherein the microcantilever has an effective surface density of tethered antibodies or antigens thereon.

4. The sensor of claim 2, wherein the microcantilever is functionalized with an antibody precursor solution having an antibody concentration of 10 μg/ml-100 μg/ml or an antigen precursor solution having an antibody concentration of 10 μg/ml-100 μg/ml.

5. The sensor of claim 2, wherein the antibody-functionalized microcantilever comprises an antibody configured to bind a pathogenic antigen or an antibody indicative of an infection.

6. The sensor of claim 5, wherein the antibody-functionalized microcantilever comprises the antibody configured to bind the pathogenic antigen and the pathogenic antigen is a SARS-CoV-2 protein.

7. The sensor of claim 5, wherein the antibody-functionalized microcantilever comprises the antigen configured to bind the antibody indicative of the infection and the antibody is indicative for a SARS-CoV-2 infection.

8. The sensor of claim 2, wherein the antibody or the antigen is conjugated to the tether by EDC-NHS chemistry.

9. The sensor of claim 2, wherein the microcantilever comprises gold.

10. The sensor of claim 2, wherein the tether is prepared from a thiocarboxylic acid precursor.

11. The sensor of claim 1 wherein the sensor comprises a field-effect transistor.

12. The sensor of claim 11, wherein the gate effect transistor is a metal-oxide-semiconductor field-effect transistor (MOSFET).

13. A system for detection of a target antigen or a target antibody in a sample, the system comprising the nanomechanical sensor according to claim 1 and a detector for detecting a response of the nanomechanical sensor to the sample and generating a signal indicative of the response of the nanomechanical sensor to the sample.

14. The system of claim 13, wherein the response is a bending moment.

15. The system of claim 13, wherein the system comprises two or more nanomechanical sensors functionalized to detect different target antigens, different target antibodies, or any combination of one or more target antibodies and one or more target antigens in the sample and wherein the detector detects the response of each nanomechanical sensor and generates a signal indicative of the response of each nanomechanical sensor.

16. The system of claim 13 further comprising a computing platform having a communication interface that receives one or more signals from the detector, and a computer in communication with the communication interface, wherein the computer comprises a computer processor and a computer readable medium comprising machine-executable code that, upon execution by the computer processor, implements a method for determining the response of the one or more nanomechanical sensors to the sample.

17. The system of claim 13 further comprising a breath collector that is operably connected to the sensor.

18. (canceled)

19. A method for the detection of a target antigen or a target antibody in a sample, the method comprising contacting the nanomechanical sensor according to claim 1 with the sample and detecting for a response of the antibody-functionalized microcantilever to the sample.

20. The method of claim 19, wherein the response to the target antigen or the target antibody is detected within 5 minutes of contacting the nanomechanical sensor with the sample.

21. The method of claim 19, wherein the sample comprises less than 10 ng/ml of the target antigen or the target antibody.

22. (canceled)

23. (canceled)

24. (canceled)

25. (canceled)

26. (canceled)

27. (canceled)
